(19) United States
(12) Patent Application Publication
(10) Pub. No.: US 2025/0025215 A1
(43) Pub. Date: Jan. 23, 2025

(54) SPINE STABILIZATION

(71) Applicant: MOVING SPINE AG, CH-8008 ZURICH (CH)

(72) Inventors: Jonas WIDMER, Fallanden (CH); Mazda FARSHAD, Zumikon (CH); Robert FRIGG, Bettlach (CH)

(73) Assignee: MOVING SPINE AG, CH-8008 ZURICH (CH)

(21) Appl. No.: 18/777,791

(22) Filed: Jul. 19, 2024

Related U.S. Application Data
(63) Continuation-in-part of application No. 18/224,679, filed on Jul. 21, 2023.

(51) Int. Cl. A61B 17/70

(52) U.S. Cl. CPC A61B 17/7053

(57) ABSTRACT

Vertebral stabilization techniques labelled “vertebropexy” can be used after microsurgical decompression (intact posterior structures) and midline decompression (removed posterior structures). Vertebropexy is a concept of semi-rigid spinal stabilization based on reinforcement of the spinal segment and is able to reduce motion, especially in flexion-extension.

### CROSS-REFERENCE TO RELATED APPLICATION

This application is a continuation-in-part of U.S. patent application Ser. No. 18/224,679, filed 21 Jul. 2023. This application is hereby incorporated by reference herein in its entirety and is made a part hereof, including but not limited to those portions which specifically appear hereinafter.

### BACKGROUND OF THE INVENTION

### Field of the Invention

The present invention relates to methods of stabilizing the spine.

### Discussion of Related Art

The biomechanical understanding of the human body is of utmost importance in orthopedic surgery and especially in spine surgery. When degeneration occurs, the stability of the disc and ligaments decreases, which can lead to instability of the segment and thus pain.

Spinal fusion has become a very common surgical procedure, among others in the treatment of degenerative disorders of the spine. The indications for this surgical procedure are diverse and include low-back pain due to facet joint osteoarthritis, degenerative spondylolistheses, degenerative scoliosis and segmental instability. The latter can also be a result of iatrogenic destabilization following surgical resection of ligamentous structures as well as the facet joint. However, spinal fusion is associated with serious long-term complications such as adjacent segment degeneration (ASD), screw loosening, pseudarthrosis, implant failure, and, in rare cases, neurovascular injury during implant insertion [1-5]. The redistribution of loads with subsequently increased biomechanical stress are believed to act as accelerators of ASD [1, 2, 6] and proximal junctional kyphosis [7]. Further, long fusions can lead to a relevant, irreversible loss of motion, which can cause postural changes [8].

Despite these problems, posterior spinal fusion (PSF) currently represents the gold standard. Alternative techniques of spinal stabilizations have not yet yielded satisfactory results with broad clinical impact. Semi-rigid fixation techniques have been proposed to overcome the above-mentioned challenges, but resulted in new complications at the implant-bone interface such as device breakage, dislocation or screw loosening [9-11]. The previous attempts at spinal stabilization also include spinous process implants. However, studies have shown higher rates of reoperation with low cost-effectiveness, which may explain why they are hardly in use anymore [9, 11]. The same applies to cervical wiring techniques, such as sublaminar wires for atlantoaxial fusion first described by Galli in 1939, which are unable to achieve sufficient stabilization.

### SUMMARY OF THE INVENTION

In conclusion, despite continued efforts to achieve appropriate spine stabilization, no technique has gained clinical acceptance. A major shortcoming of the known techniques is insufficient stabilization of the spine, in particular in the long term. A further shortcoming is that the known techniques frequently lead to significant, sometimes complete, immobilization of the segment, in particular in clinically important directions such as flexion-extension and shear movement. Therefore, there is a need to advance the state of the art with respect to stabilization of the spine.

The present invention is aimed at providing a method to stabilize the spine of a patient in need thereof, while at least partially avoiding the complications associated with the known techniques, particularly the implant- and fusion-related complications. In particular, it is an object of the present disclosure to achieve targeted stabilization of the spine without immobilizing the segment. With respect to the directions of motion, the present disclosure aims to achieve passive stability in clinically important directions of motion such as flexion-extension and shear movements, but without stiffening other directions of motion. It is a further object to provide a reversible method of spine stabilization. It is a further object for at least some embodiments to improve the biological compatibility of the implanted material used in the method, e.g., by enhancing the biological integration of the implanted material into the surrounding tissue.

According to the present disclosure, these objects are addressed by the features of the independent claims. In addition, further advantageous embodiments follow from the dependent claims and the description.

The present disclosure relates to a method of stabilizing the spine of a patient in need thereof.

The method comprises the step of providing a first vertebra having a first fastening edge and a second vertebra having a second fastening edge, wherein the first fastening edge and the second fastening edge are arranged opposite each other and facing away from each other.

The method further comprises the step of providing a strap extending in a longitudinal direction from a front section to a rear section.

The method further comprises the step of passing the front section of the strap around the first fastening edge, then from the first fastening edge to the second fastening edge and then around the second fastening edge.

The method may further comprise the optional step of optionally passing the front section of the strap from the second fastening edge to the first fastening edge and repeating step c).

The method further comprises the step of tensioning the strap and fastening the front section to the rear section such that the first vertebra and the second vertebra are fixated relative to each other.

The method of the present disclosure may be labelled as “vertebropexy” and it constitutes a new concept of semi-rigid spinal stabilization based on reinforcement of the spinal segment using a strap, such as a ligament, a synthetic material or a hybrid material comprising a ligament component and a synthetic components. Vertebropexy not only restores the native segmental stability after decompression, but also transfers the segment to a semi-rigid state.

The method of the present disclosure and the variants disclosed herein have a range of advantages. They allow a targeted stabilization of the spine to counteract degeneration-related or iatrogenic (e.g., decompression) instability, but without immobilizing the segment. Depending on the application, native stability of the segment may be restored fully or partially after surgical decompression and the segment may be placed in a more stable state, without complete immobilization. Depending on the application, lumbar segmental motion may be significantly reduced, especially in flexion-extension and, though typically to a lesser extent, in shear motion. Depending on the variant of the method being used, all other directions of motion may remain flexible and correspond to the preoperative range of motion. The method of the present disclosure may preferably be reversible, thereby still allowing traditional techniques such as spinal fusion to be performed subsequently. The method may also be carried out using no foreign material, e.g., using ligaments or allografts, which enhances the biocompatibility and may allow the implanted material to grow together with adjacent tissue. Furthermore, the method of the present disclosure may be used for different indications: for example, an interspinous variant can be used after microsurgical decompression if the surgeon wishes to achieve more stability, such as in existing low-grade spondylolisthesis. When the posterior structures are omitted, such as after midline decompression, a spinolaminar variant can be used. Finally, the technique disclosed herein allow for an easy handling by the surgeon.

The strap may, for example, be able to undergo deformations elastically, without permanent damage. Depending on the application, the strap may be hyperelastic. Additionally, or alternatively, depending on the application, the strap may be viscoelastic.

Depending on the application, the strap may have different mechanical properties. These mechanical properties may be measured using different measurement protocols. The variants of the strap described in this paragraph refer to the mechanical properties when measured as described in the third example below. As an example, the strap may have an ultimate load of at least 1000 N, such as at least 1200 N. In some variants, the strap has an ultimate load from 1000 N to 5000 N. The ultimate load is typically defined as the maximum load that the strap can endure without tearing. In some variants, the strap may have a linear stiffness from 100 N/mm to 520 N/mm, preferably from 370 N/mm to 470 N/mm. Additionally or alternatively, the strap may have an ultimate displacement of at least 6 mm, preferably from 7 mm to 14 mm. The parameters described in this paragraph refer to a strap length of 95 mm.

The mechanical properties of the strap may also be provided independently of the specific measurement protocol used in the third example. This paragraph describes the mechanical properties of some variants of the strap, referring specifically to the strap itself in a linear configuration, before any loops are formed. Thus, for example, when determining the maximum load of the strap as described in this paragraph, the load force is applied to the two opposite ends of the strap, as in many conventional load tests. In some variants, the strap has an ultimate load of at least 425 N, preferably from 500 N to 1250 N, more preferably from 600 N to 900 N. The ultimate load is typically defined as the maximum load that the strap can endure without breaking or tearing. In some variants, the strap may have a linear stiffness from 80 N/mm to 130 N/mm, preferably from 92.5 N/mm to 117.5 N/mm. Additionally or alternatively, the strap may have an ultimate displacement of at least 1.5 mm, preferably from 1.75 mm to 3.5 mm. The parameters described in this paragraph may e.g. refer to a strap length of 95 mm.

In some variants, step e) includes the steps of: tensioning the strap such that the first vertebra and the second vertebra are fixated relative to each other; fastening the front section to the rear section while the strap is being tensioned.

Depending on the application, the steps of tensioning and fastening may be carried out essentially simultaneously, or with a slight delay relative to each other, or subsequently after one another. If they are carried out in a subsequent fashion, the steps may be carried out directly after one another, or with one or more optional intermediate steps. In some variants, the step of tensioning is carried out before the step of fastening. In other variants, the step of tensioning is carried out after the step of fastening.

The method of the present disclosure includes the step of c) tensioning the strap. The tensioning may, for example, lead to the first vertebra and the second vertebra being positioned relative to each other. In some variants, the first vertebra and the second vertebra may be at least partially fixated relative to each other. Depending on the application, the strap may be tensioned in different ways and with various effects. For example, the strap may be tensioned such that the first vertebra and the second vertebra are fixated relative to each other with respect to at least one direction of motion. In other words, motion of the first vertebra relative to the second vertebra may at least partially be restricted in at least one direction of motion. Depending on the field of application, one or more directions of motion may be affected. In some variants, relative movement between the first vertebra and the second vertebra may be at least partially restricted in one or more of the following directions of motion: flexion, extension, lateral bending, axial rotation, anteroposterior shear and lateral shear. The relative movement in the respective directions of motion may be restricted to different degrees. As an example, in one variant, the range of motion in flexion and/or extension is decreased by at least 50%, preferably by at least 60%, compared to the native state. The native state describes the state of the first vertebra and the second vertebra before performing the method disclosed herein.

Depending on the application, one of the first vertebra and the second vertebra may be labelled as a cranial vertebra and the other of the first vertebra and the second vertebra may be labelled as a caudal vertebra. The first vertebra and the second vertebra are typically adjacent to each other, specifically directly adjacent to each other. In some variants, the first vertebra and the second vertebra are interspaced by at least one intermediate vertebra, preferably by one intermediate vertebra or two intermediate vertebrae, more preferably by one intermediate vertebra.

Depending on the application, the step of providing the first vertebra and the second vertebra may include one or more preparatory surgical steps. The preparatory surgical steps may include a laminotomy, optionally followed by flavectocomy, optionally followed by recessotomy. Additionally, or alternatively, the preparatory surgical steps may include removal of supraspinous and interspinous ligaments. Additionally, or alternatively, the preparatory surgical steps may include at least partial removal of the spinous processes. Depending on the application, the first vertebra may already include the first fastening edge in its native state, i.e. prior to performing the method. Additionally, or alternatively, the second vertebra may already include the second fastening edge in its native state. In further variants, it may be necessary to provide the first fastening edge and/or the second fastening edge, e.g., by means of a surgical step such as boring, drilling or cutting. As an example, step a) may include drilling a hole in the spinous process of the first vertebra and/or the second vertebra. The hole in the spinous process may e.g., have a diameter from 2 mm to 10 mm, preferably from 4 mm to 6 mm. Drilling the hole may include pre-drilling a pre-hole and then overdrilling the pre-hole to provide the hole. The pre-hole may for example have a diameter from 1 mm to 5 mm, preferably from 3 mm to 3.5 mm.

In some variants, the method includes drilling or hole and/or providing a tunnel in the interspinous ligament. For example, a hole or tunnel may be provided in the interspinous ligament adjacent in cranial direction to the cranial edge of the cranial vertebra and/or in the interspinous ligament adjacent in caudal direction to the caudal edge of the caudal vertebra.

When carrying out the method of the present disclosure, typically, access is provided to a proximal side of the first vertebra and the second vertebra. Depending on the application, the proximal side may for example be a dorsal side of the vertebrae and the distal side may be oriented towards the vertebral foramen. Step c) may include passing the front section of the strap from a proximal side around the first fastening edge to a distal side, then on the distal side from the first fastening edge to the second fastening edge and then from the distal side around the second fastening edge to the proximal side. Optionally, step d) may then include passing the front section of the strap on the proximal side from the second fastening edge to the first fastening edge and repeating step c).

Depending on the application, the front section may be fastened to the rear section in different ways. Typically, the front section is connected to the rear section such that it directly contacts the rear section. In some variants, the fastening includes at least one of knotting, suturing, sewing, stitching, knitting or felting the front section to the rear section. When the fastening involves knotting, typically, two or more knots are knotted, preferably from four to seven knots. Preferably, the fastening includes suturing or knotting the front section to the rear section. In some variants, the front section is fastened to the rear section such that the front section and the rear section are approximated towards each other, which may involve application of a tensile force to the front section in a direction towards the rear section and application of a tensile force to the rear section in a direction towards the front section. In some variants, the front section and the rear section may contact each other after fastening. In some variants, the front section and the rear section are interspaced from each other after fastening. For example, after fastening, the front section and the rear section may be interconnected to each other but interspaced from each other. For example, in some variants, after fastening, the front section and the rear section are interconnected to each other through a connector, e.g. a self-tightening connected. In some variants, after fastening, the front section and the rear section are interspaced from each other by the connector. Thus, in some variants, at least a portion of the connector may for example extend between the front section and the rear section after fastening.

In some variants, the front section is fastened to the rear section using a connector having self-tightening properties. For example, the connector may comprise in a mounted position a first hitch formed by a suture encompassing the front section, and a second hitch formed by a suture encompassing the rear section. The suture forming the first hitch and the suture forming the second hitch may be interconnected to each other between the first hitch and the second hitch. The first hitch and/or the second hitch may for example comprise a suture loop which in the mounted position encompasses the first respectively the second tendon segment in at least two turns. For example, in some variants, the self-tightening connector may for example comprise one or more tension knots, which may e.g. be applied to the front section and rear section. In an embodiment, the first tension knot and the second tension knot are Prusik knots.

As explained further detail below, in some variants a front fastening fiber and/or a rear fastening fiber may be connected to the main body (e.g. by a SpeedWhip). In these variants, the front fastening fiber and the rear fastening fiber may then optionally be interconnected to each other. These variants may e.g. be used to bridge a gap between the front section and the rear section of the strap.

The strap may be made of different materials. For example, the strap may comprise a synthetic material, a ligament or a tendon, preferably a tendon. The ligament may, for example, be an autograft, an allograft or a xenograft. Preferably, the ligament is an allograft. The synthetic material may for example be braided and/or woven and/or stitched and/or felted. The synthetic material may e.g. be or comprise a tape. In some variants, the synthetic material is a non-metallic cerclage. When using a non-metallic cerclage, for example, an Arthrex FiberTape Cerclage may be used. Depending on the application, the non-metallic cerclage may be braided or woven from a polyblend of ultra high molecular weight polyethylene and polyester materials. In some variants, the non-metallic cerclage is made from pure polyethylene terephthalate (PET). Additionally, or alternatively, the non-metallic cerclage may be a flat braided suture or a woven suture. In some variants, the strap is made of a woven structure or a tape or a synthetic ligament.

One advantage of using a ligament or a tendon is that it ensures biological compatibility and allows the strap to grow together with surrounding tissue. One advantage of including a synthetic component is that synthetic components are typically widely available and relatively cheap compared to biological material.

In some variants, the strap is made of a hybrid material, comprising a synthetic component and a natural component. For example, in some variants, the strap comprises a synthetic component and a ligament component (e.g. an autograft component, an allograft component, or a xenograft component). Depending on the application, the components of the hybrid material may have different arrangements with respect to each other. For example, the synthetic component and the ligament (or tendon) component may extend essentially parallel and adjacent to each other. For example, in some variants, one of the materials forms a sheath and circumferentially encompasses at least a longitudinal section of the other component. As an example, the synthetic component may form a sheath circumferentially encompassing at least a longitudinal section of the ligament (or tendon) component. This variant may e.g. be labelled as an “around/inside arrangement”.

In some variants, the components of the hybrid material are arranged in a side-by-side configuration. For example, in some variants, the synthetic component and the ligament (or tendon) component extend parallel and next to each other in longitudinal direction. In some variants, the synthetic component and the ligament (or tendon) component contact each other in longitudinal direction, such that a longitudinal section of an outer surface of the synthetic component and a longitudinal section of an outer surface of the ligament (or tendon) component form together an outer surface of the hybrid material. In some variants, the synthetic component and the ligament (or tendon) component contact each other along a length of the synthetic component or along a length of the ligament (or tendon) component. In some variants, the synthetic component is arranged on and contacts a first lateral side of the ligament (or tendon) component. For example, the synthetic component may be stitched onto the ligament (or tendon) component, for example onto the first lateral side of the ligament (or tendon) component.

Depending on the application, the strap may be made of a hybrid material along its entire length, or only for a longitudinal section of the strap. As an example, in some variants, the strap is made of a hybrid material in a first longitudinal section of the strap and of a ligament (or tendon) in a second longitudinal section of the strap. Other arrangements may also be envisioned. For example, the hybrid material may be strategically placed to reinforce areas of wear and tear. Depending on the application, the components of the hybrid material may either be fixated with respect to each other (e.g. by suturing them together), or they may be displaceable with respect to each other.

Depending on the application, the strap may or may not comprise additional components in addition to a main body. For example, the strap may comprise reinforcement elements connected to the main body. As an example, the reinforcement elements may be arranged at positions of the strap that contact the vertebrae, in particular positions that contact the fastening edges. As an example, each position of the strap that is passed around the first and/or second fastening edge may in some variants be reinforced.

Additionally, or alternatively, the strap may comprise a front fastening fiber connected to a main body of the strap in or near the front section and a rear fastening fiber connected to the main body of the strap in or near the rear section. Optionally, the rear fastening fiber may form a loop configured for receiving the front fastening fiber. The front fastening fiber and/or the rear fastening fiber may, for example, each be connected to the main body of the strap by a stitch, preferably a Krackow or Baseball stitch. In some variants, the front fastening fiber and/or the rear fastening fiber are each connected to the main body of the strap by a SpeedWhip. In some variants, the front fastening fiber and/or the rear fastening fiber are each connected to the main body of the strap by a knot, an anchor or a button.

Typically, the front fastening fiber and/or the rear fastening fiber are each connected to the main body of the strap in a tensile-resistant manner. Optionally, after tensioning the strap, a gap between the front section and the rear section of the strap may be bridged e.g. by the front fastening fiber and/or the rear fastening fiber, as explained above.

The front fastening fiber and the rear fastening fiber may for example serve to facilitate the step of tensioning the strap. In an embodiment, the step of tensioning the strap comprises creating a tension knot between the front fastening fiber and the rear fastening fiber, tensioning at least the front fastening fiber and tightening the tension knot. Optionally, the step of tensioning the strap may include applying opposite tensile forces to the front fastening fiber and the rear fastening fiber.

The method disclosed herein may be performed in different vertebrae and different sections, regions or areas of the vertebrae.

In a first variant, which may be labelled as “interlaminar vertebropexy”, one of the two vertebrae is a cranial vertebra and the other of the two vertebrae is a caudal vertebra, wherein the fastening edge of the cranial vertebra is a cranial edge of the lamina and the fastening edge of the caudal vertebra is a caudal edge of the lamina. In this variant, for example, the distance in the longitudinal direction between a front end of the front section and a front end of the rear section of the strap in a relaxed state may range from 120% to 180%, preferably from 140% to 160%, more preferably 150%, of the distance between the first fastening edge and the second fastening edge.

In a second variant, which may be labelled as “interspinous vertebropexy”, one of the two vertebrae is a cranial vertebra and the other of the two vertebrae is a caudal vertebra, wherein the fastening edge of the cranial vertebra is arranged on the spinous process of the cranial vertebra and the fastening edge of the caudal vertebra is arranged on the spinous process of the caudal vertebra. In this variant, for example, the distance in the longitudinal direction between a front end of the front section and a front end of the rear section of the strap in a relaxed state may range from 300% to 500%, preferably from 350% to 450%, more preferably from 380% to 420%, of the distance between the first fastening edge and the second fastening edge.

In a third variant, which may be labelled as “spinolaminar vertebropexy”, one of the two vertebrae is a cranial vertebra and the other of the two vertebrae is a caudal vertebra. In one variant, the fastening edge of the cranial vertebra is a cranial edge of the lamina of the cranial vertebra and the fastening edge of the caudal vertebra is arranged on the spinous process of the caudal vertebra. For example, the fastening edge of the caudal vertebra may in this case e.g. be a caudal edge of the spinous process of the caudal vertebra, or a hole drilled in the spinous process of the caudal vertebra. In a further variant, the fastening edge of the caudal vertebra is a caudal edge of the lamina of the caudal vertebra and the fastening edge of the cranial vertebra is arranged on the spinous process of the cranial vertebra. For example, the fastening edge of the cranial vertebra may in this case e.g. be a cranial edge of the spinous process of the cranial vertebra, or a hole drilled in the spinous process of the cranial vertebra.

Depending on the application, different positions on the spinous process may be used for the respective fastening edge, in particular in the interspinous vertebropexy variant and in the spinolaminar vertebropexy variant. For example, natural edges of the spinous process may be used as fastening edges (such as a cranial or caudal edge of the spinous process), or synthetic edges may be created and used as fastening edges, which may e.g. involve drilling a hole through the spinous process or other parts of the vertebra or vertebrae. In some variants, the fastening edge of the cranial vertebra is a hole drilled in the spinous process of the cranial vertebra. In some variants, the fastening edge of the cranial vertebra is a cranial edge of the cranial vertebra, such as e.g. a cranial edge of the spinous process of the cranial vertebra or a cranial edge of the lamina of the cranial vertebra. Alternatively, or in combination, in some variants, the fastening edge of the caudal vertebra is a hole drilled in the spinous process of the caudal vertebra. In some variants, the fastening edge of the caudal vertebra is a caudal edge of the caudal vertebra, such as e.g. a caudal edge of the spinous process of the caudal vertebra or a caudal edge of the lamina of the caudal vertebra.

Cranial edge of the cranial vertebra as used herein may for example relate to edges on the cranial vertebra that face in the cranial direction, e.g. that face towards the brain. Caudal edge of the caudal vertebra as used herein may for example relate to edges on the caudal vertebra that face in the caudal direction, e.g. that face towards the feet.

It is understood that the different variants of the fastening edge arranged on the spinous process may be selected independently of each other for the cranial and caudal vertebra. Thus, if for example the fastening edge of the caudal vertebra is a caudal edge of the caudal vertebra, then the fastening edge of the cranial vertebra may be independently selected to be e.g. a cranial edge of the cranial vertebra, or a hole drilled in the spinous process of the cranial vertebra.

It is understood that the different variants of the fastening edge, in particular the different variants relating to the fastening edge arranged on the spinous process, may be selected independently for each repetition of the step c) (namely of the step of passing the front section of the strap around the first fastening edge, then from the first fastening edge to the second fastening edge and then around the second fastening). Thus, in some variants, the first fastening edge is the same in each repetition of step c). Alternatively, or in combination, in some variants, the second fastening edge is the same in each repetition of step c). Alternatively, or in combination, in some variants, the first fastening edge changes upon repeating step c). Alternatively, or in combination, in some variants, the second fastening edge changes upon repeating step c).

Selected examples which involve one repetition of step c) (i.e. in total two iterations of step c)) may e.g. be described using the nomenclature FFE-1→SFE-1→FFE-2→SFE-2, where FFE-1 denotes the first fastening edge of the first iteration of step c), SFE-1 denotes the second fastening edge of the first iteration of step c), FFE-2 denotes the first fastening edge of the second iteration of step c) and SFE-2 denotes the second fastening edge of the second iteration of step c). Following this nomenclature, selected examples among other examples include:
hole in cranial spinous process→hole in caudal spinous process→hole in cranial spinous process→hole in caudal spinous process;
cranial edge of cranial spinous process→caudal edge of caudal spinous process→cranial edge of cranial spinous process→caudal edge of caudal spinous process;
hole in cranial spinous process→hole in caudal spinous process→cranial edge of cranial spinous process→caudal edge of caudal spinous process;
hole in cranial spinous process→caudal edge of caudal spinous process→hole in cranial spinous process→caudal edge of caudal spinous process;
hole in cranial spinous process→caudal edge of caudal spinous process→cranial edge of cranial spinous process→hole in caudal spinous process;
cranial edge of cranial spinous process→hole in caudal spinous process→hole in cranial spinous process→caudal edge of caudal spinous process;
cranial edge of cranial spinous process→hole in caudal spinous process→cranial edge of cranial spinous process→hole in caudal spinous process;
cranial edge of cranial spinous process→caudal edge of caudal spinous process hole in cranial spinous process→hole in caudal spinous process;
hole in cranial spinous process→caudal edge of caudal spinous process→cranial edge of cranial spinous process→caudal edge of caudal spinous process;
cranial edge of cranial spinous process→hole in caudal spinous process→cranial edge of cranial spinous process→caudal edge of caudal spinous process;
cranial edge of cranial spinous process→caudal edge of caudal spinous process→hole in cranial spinous process→caudal edge of caudal spinous process;
cranial edge of cranial spinous process→caudal edge of caudal spinous process→cranial edge of cranial spinous process→hole in caudal spinous process;
cranial edge of cranial spinous process→hole in caudal spinous process→hole in cranial spinous process→hole in caudal spinous process;
hole in cranial spinous process→caudal edge of caudal spinous process→hole in cranial spinous process→hole in caudal spinous process;
hole in cranial spinous process→hole in caudal spinous process→cranial edge of cranial spinous process→hole in caudal spinous process; and
hole in cranial spinous process→hole in caudal spinous process→hole in cranial spinous process→caudal edge of caudal spinous process;

It is understood that these are only a selection of possible variants, with the method of the present invention also encompassing further variants not specifically mentioned in the list of the previous paragraph. It is also understood that when step c) is repeated twice or more times (i.e. the method involves a total of three or more iterations of step c)), the fastening edges may or may not change, similar to the variants outlined above or one repetition of step c) (i.e. a total of two iterations of step c)).

In some variants, step c) is repeated at least once, thereby forming at least a first and a second loop. For example, the first loop may be defined in a first iteration of step c), and the second loop may be defined in a second iteration of step c). In some variants, step c) is repeated exactly once, thereby forming a total of two loops. In some variants, step c) is repeated twice or even more than two times, thereby forming a total of at least three loops.

Depending on the application, the first and second loop (and optional further loops) may have different arrangements relative to each other. In some variants, in a cross-section parallel to the coronal plane of the patient, the first loop crosses the second loop at at least one crossing position. In some variants, at least a section of the first loop and at least a section of the second loop are arranged adjacent to each other in a cross-section parallel to a coronal plane of the patient. In some variants, at least a section of the first loop and at least a section of the second loop contact each other. In some variants, the front section and the rear section of the strap are arranged on an outer of the two sections facing away from the spinous process. In further variants, the front section and the rear section of the strap are arranged on an inner of the two sections facing towards the spinous process.

In some variants, the strap crosses itself at one or more crossing positions selected from one or more of the following: a hole in the spinous process of the first vertebra, a hole in the spinous process of the second vertebra, or a hole in the interspinous ligament between the first and second vertebrae.

Depending on the application, the first and second loop may be formed independently of each other. For example, the first fastening edge of the first loop may be chosen independently of the first fastening edge of the second loop. Thus, the first fastening edge of the first loop and the first fastening edge of the second loop may be the same or different. Similarly, the second fastening edge of the second loop may be chosen independently of the second fastening edge of the second loop. Thus, the second fastening edge of the first loop and the second fastening edge of the second loop may be the same or different.

In some variants, the at least two loops may optionally be interconnected to each other, e.g. fixated to each other. For example, the first and second loop may optionally be stitched together. In some variants, the first loop and the second loop are interconnected to each other at an interconnection position arranged in a spinal direction of extension between the spinous process of the cranial vertebra and the spinous process of the caudal vertebra. Depending on the application, different loop interconnection positions may be selected. For example, the loop interconnection position may be chosen on either lateral side of the spinous processes of the cranial and caudal vertebrae. In some variants, the loop interconnection position is chosen such that it is arranged on the same lateral side of the spinous processes of the cranial and caudal vertebrae as a fastening position at which the front section is fastened to the rear section. Alternatively, or in combination, the loop interconnection position may be chosen such that it is arranged on an opposite lateral side of the spinous processes of the cranial and caudal vertebrae as a fastening position at which the front section is fastened to the rear section.

Depending on the application, the strap may be passed between the first vertebra and the second vertebra in different ways. For example, when the strap is passed from the first fastening edge to the second fastening edge, a direct route may be chosen, or the strap may optionally be guided around intermediate structures along the way. It is also possible for the strap to cross from a first lateral side to an opposite second lateral side.

In some variants, the front section of the strap is passed from a first lateral side of the spinous processes to an opposite second lateral side of the spinous process of the first and second vertebra during one or more of the following steps: passing the front section of the strap from the first fastening edge to the second fastening edge; and/or passing the front section of the strap from the second fastening edge to the first fastening edge.

The first lateral side and the second lateral side are defined by the spinous processes of the first vertebra and the second vertebra. For example, the first lateral side and the second lateral side may be separated by the median plane of the patient, which extends along a direction of extension of the spine.

As a result of passing the strap from the first lateral side to the second lateral side, traverse positions may e.g. be formed. In some variants, after fastening the strap, the strap may comprise one or more traverse positions each arranged between the first fastening edge and the second fastening edge, wherein at each traverse position, the strap passes from a first lateral side to an opposite second lateral side of the spinous process of the first and second vertebra. In some variants, after fastening the strap, the strap comprises at least two traverse positions.

Depending on the application, the strap may in some variants cross itself in different ways. For example, the strap may e.g. be guided through the same hole in a spinous process twice and in such a fashion that it crosses itself. In some variants, the strap may cross itself in a space arranged between the spinous process of the first and second vertebra.

In some variants, an intermediate section of the strap arranged in longitudinal direction of the strap between the front section and the rear section forms a loop. In some variants, the loop formed by the intermediate section of the strap is arranged between the spinous process of the first vertebra and the spinous process of the second vertebra. The strap may then optionally be passed through that loop. In some variants, the method comprises passing the front section or the rear section of the strap through the loop formed by the intermediate section of the strap.

Depending on the application, the fastening edges may optionally be reinforced. For example, where a hole in the spinous process is used as fastening edge, a reinforcement sleeve or tunnel (e.g. a metal sleeve) may be applied to the hole. It is possible to apply a first reinforcement sleeve to a hole in the spinous process of the first (e.g. cranial) vertebra and/or a second reinforcement sleeve may be applied to a hole in the spinous process of the second (e.g. caudal) vertebra. The sleeve may for example be cylindrical and may have an oval cross-section, preferably a circular cross-section. Typically, an outer diameter of the reinforcement sleeve is selected such that it matches an inner diameter of the respective hole. In some variants, the outer diameter of the reinforcement sleeve may even slightly exceed an inner diameter of the respective hole in order to achieve a press-fit.

Reinforcement structures other than reinforcement sleeves may also be used. For example, a reinforcement support may optionally be applied to a cranial edge of the cranial vertebra and/or to a caudal edge of the caudal vertebra. The reinforcement support may e.g. have a curved structure matching the respective cranial edge respectively the respective caudal edge.

Typically, the reinforcement structure has rounded edges to minimize damage to the strap, especially under tension. In some variants, the reinforcement structure has a smooth surface to facilitate gliding of the strap along its surface. It is understood that it is possible to use more than one reinforcement structure. It is also possible to use more than one type of reinforcement structure discussed herein.

Depending on the application, the strap may be applied such that tensile forces are applied to the vertebrae in different directions. For example, after fastening the front section to the rear section, the strap may exert a tensile force on the first vertebra and on the second vertebra in a tensile force direction that is essentially in parallel to the direction of extension of the spine between the first vertebra and the second vertebra. This direction may, for example, be used for interspinous vertebropexy or intervertebral vertebropexy. In a further variant, after fastening the front section to the rear section, the strap may also exert a tensile force on the first vertebra and on the second vertebra in a tensile force direction that includes a vector component in the dorsal direction. This direction may, for example, be used for spinolaminor vertebropexy. These variants may be used for different applications, depending on the needs of the patient. For example, providing a tensile force that is essentially in parallel to the direction of extension of the spine may lead to a particularly high stabilization of flexion-extension while still allowing mobility in other directions of movement, in particular shear movements. On the other hand, providing a tensile force that includes a vector component in the dorsal direction typically has a greater effect on shear motion.

Depending on the application, it may be desirable to provide further structures to influence, stabilize or alter the relative positions of the strap with respect to the first vertebra and the second vertebra, in particular the relative position after tensioning and fastening of the strap. In some variants, the method includes applying to the strap a positional securing element configured for securing a position of the strap with respect to displacement in the ventral or dorsal direction. The positional securing element may, e.g., interconnect two sections of the strap arranged on opposite lateral sides of the spinous process of the two vertebrae. Alternatively, or in combination, the positional securing element may interconnect the strap to a body structure arranged further in the ventral direction than the strap. The body structure arranged further in the ventral direction may e.g. be one or more of the following: an inferior articular process of the ventral vertebra, a transverse process of the ventral vertebra and/or of the caudal vertebra; and/or a superior articular process of the ventral vertebra and/or of the caudal vertebra.

Depending on the application, it may be desirable to distribute at least a portion of the tension to other structures, e.g. other body structures such as upper or lower sections of the spine. This may for example be desirable to lower the tension load exerted on the first vertebra and/or the second vertebra. For example, excessive loads on the spinous process may lead to physical damage to the spinous process. In some variants, the method includes interconnecting the strap to a tension relief body structure through a tension distribution structure, such that at least a portion of the tension is distributed from the first and/or second fastening edge to the tension relief body structure. The tension relief body structure may e.g. be another part of the spine arranged in the cranial direction above the cranial vertebra or arranged in the caudal direction below the caudal vertebra. For example, the tension relief body structure may in some variants be the superior articular process of the cranial vertebra. Depending on the application, the tension distribution structure may e.g. be interconnected to a section of the strap that contacts the first fastening edge or the second fastening edge. The tension distribution structure may for example have a tensile strength that is at least the same as or higher than the tensile strength of the strap. In some variants, the tensile strength of the tension distribution structure is higher than that of the strap. The tension distribution on structure may e.g. be a further strap, e.g. a ligament, a tendon, or a synthetic construct.

Depending on the application, the method disclosed herein may be performed once, twice or even more than two times on the patient. The same vertebrae could be involved, or different vertebrae could be involved. Performing the method twice or more than two times could e.g. be used to enhance spine stabilization. Therefore, in some variants, the present disclosure relates to a method of enhancing stabilization of the spine of a patient in need thereof, the method comprising: performing the method according to any one of the embodiments described herein on the patient to form a first looping implant; and performing the method according to any one of the embodiments described herein on the same patient to form a second looping implant.

In some variants, for the first looping implant, the fastening edge of the cranial vertebra is a cranial edge of the lamina and the fastening edge of the caudal vertebra is a caudal edge of the lamina. For the second looping implant, the fastening edge of the cranial vertebra may e.g. be arranged on the spinous process of the cranial vertebra and the fastening edge of the caudal vertebra may e.g. be arranged on the spinous process of the caudal vertebra.

Depending on the integrity of the dorsal structures after decompression, different variants may be used. As an example, the variant labelled “interspinous vertebropexy” may be used following posterior microsurgical decompression with preservation of midline structures. Additionally, or alternatively, the variant labelled “interlaminar vertebropexy” may be used following posterior decompression without preservation of midline structures. These structures mostly provide passive stability in flexion.

The variant labelled as “spinolaminar vertebropexy” allows for a range of advantages. Among them, this variant affects shear forces to a particularly great extent. Without wishing to be bound to a theory, this could possibly be explained by the fact that the fixation of the segment with the spinolaminar technique happens not purely in the cranio-caudal direction, but also in the antero-posterior direction and can thus absorb forces in this direction. Further advantages include an increase in a-p stability and easy tensioning with designated tensioning systems.

The tensioning and fastening may be performed in different ways and using different forces. In some variants, during the step of tensioning the strap, a tension force from 40 N to 200 N, preferably from 50 N to 100 N, more preferably from 50 N to 70 N, is applied. In some variants, a tension force from 40 N to 90 N, preferably from 50 N to 70 N, is applied. The fastening may include knotting a knot. In further variants, the step of fastening the front section to the rear section may include overlapping the front section with the rear section and connecting the front section with the overlapped rear section. Typically, the front section is overlapped with the rear section such that the front section and the overlapped rear section extend in the same or in opposite directions. Depending on the application, the front section may for example be connected with the overlapped rear section by suturing, knitting or felting, preferably suturing. The region of overlap may, for example, have a length from 5 mm to 35 mm. In some variants, the front section is fastened to the rear section at a fastening position arranged in a spinal direction of extension between the spinous process of the first vertebra and the spinous process of the second vertebra. The fastening position may e.g. face in the dorsal direction or in the ventral direction.

One advantage of the method of the present disclosure is that it allows for durable mechanical stabilization of the spine. For example, when the front section and the rear section are overlapped in opposing directions, the front section and the rear section may continue to extend along their previous directions, and there is no need to form a folded edge to join the two ends together, which would be necessary if the front section and the rear section were inserted into a clamping element serving to connect the two sections. Since no folded edge is formed, the risk of generating a crack or tear in the strap is minimized.

A further advantage is that in its implanted stage, the strap may only occupy a small volume and therefore constitute a minimal intrusion to the body. This is possible because the strap is first tensioned and then the front section is fastened to the rear section. As an example, at least in some variants, it is not necessary to use an additional clamping element that clamps the front section and the rear section in order to fasten them. Rather, it is possible, for example, to knot, suture, sew, stitch, knot or felt the front section to the rear section. Thereby, no additional body volume is occupied by an additional clamping element. In some variants, an additional clamping element may be used. Depending on the application, the strap may have different geometries. In some variants, at a position of the strap in which the front section has been fastened to the rear section, the strap has a maximum width of up to 20 mm, preferably up to 15 mm, more preferably up to 10 mm, even preferably up to 8 mm, even more preferably up to 6 mm, in at least one of the following directions: dorsal direction or lateral direction. For example, the dorsal extension may be less than 6 mm. Additionally or alternatively, the area occupied by the strap in a cross sectional area orthogonal to the direction of extension of the spine between the first vertebra and the second vertebra may, for example, not exceed 100 mm², preferably may not exceed 60 mm², more preferably may not exceed 40 mm².

Depending on the application, the method also allows to generate straps which, in the implanted stage, exhibit an outer shape with a gradual outer transition between the section in which the front section is fastened to the rear section (e.g. by overlapping and suturing them), and the adjacent sections in which the front section is not fastened to the rear section (e.g. where the front section does not contact the rear section). For example, at a position of the strap in which the front section has been fastened to the rear section, the area occupied by the strap in a cross section orthogonal to the direction of extension of the spine between the first vertebra and the second vertebra may be less than 300%, preferably less than 250%, more preferably less than 200% of the cross sectional area occupied by the strap at a position adjacent to the position in which the front section has been fastened to the rear section.

In some variants, the front section of the strap and the rear section of the strap do not overlap with each other after tensioning. For example, a gap may be formed between the front section and the rear section after tensioning. For example, the strap may be chosen to have a length such that after looping and tensioning, a gap remains. This gap may e.g. be covered by a tape. In some variants, the gap is bridged by a front fastening fiber and a rear fastening fiber being interconnected to each other, as described herein. The front fastening fiber and the rear fastening fiber may e.g. be interconnected through a clamp, a knot, a button or another interconnection element.

### BRIEF DESCRIPTION OF SEVERAL VIEWS OF THE DRAWINGS

The invention described herein will be more fully understood from the detailed description given herein below and the accompanying drawings, which should not be considered limiting to the invention described in the appended claims.

FIG. 1 shows a visualization of the allograft preparation for both interlaminar vertebropexy (left) and interspinous vertebropexy (right) and determination of the graft length for the studies described in example 1;

FIG. 2 shows illustrations of the interspinous vertebropexy in which the allograft is looped through the holes in the spinous processes and then tightened, as described in example 1;

FIG. 2A shows one illustration in a series of the interspinous vertebropexy in which the allograft is looped through the holes in the spinous processes, as described in example 1;

FIG. 2B shows another illustration in the series of the interspinous vertebropexy in which the allograft is looped through the holes in the spinous processes, as described in example 1;

FIG. 2C shows another illustration in the series of the interspinous vertebropexy in which the allograft is looped through the holes in the spinous processes, as described in example 1;

FIG. 2D shows another illustration in the series of the interspinous vertebropexy in which a tension knot is applied, as described in example 1;

FIG. 2E shows another illustration in the series of the interspinous vertebropexy in which a tension is applied, as described in example 1;

FIG. 2F shows another illustration in the series of the interspinous vertebropexy in which the loose end of the allograft is fixed with the other end, as described in example 1;

FIG. 3 shows illustrations of the interlaminar vertebropexy in which two allografts are passed behind the laminae and brought together to form a loop and are then tightened, as described in example 1;

FIG. 3A shows one illustration in a series of the interlaminar vertebropexy in which a first allografts is passed behind the laminae, as described in example 1;

FIG. 3B shows another illustration in the series of the interlaminar vertebropexy in which the two ends of the first allograft are connected with a tension knot, as described in example 1;

FIG. 3C shows another illustration in the series of the interlaminar vertebropexy in which the knot is tightened, as described in example 1;

FIG. 3D shows another illustration in the series of the interlaminar vertebropexy in which the procedure is repeated for the other side, as described in example 1;

FIG. 4 shows illustrations of the setup for biomechanical testing used in example 1 (FIG. 4A), illustration of the interspinous vertebropexy (FIG. 4B) and of the interlaminar vertebropexy (FIG. 4C), as described in example 1;

FIG. 4A shows an illustration of the setup for biomechanical testing used in example 1;

FIG. 4B shows an illustration of the interspinous vertebropexy used in example 1;

FIG. 4C shows an illustration of the interlaminar vertebropexy as described in example 1;

FIG. 5 shows an illustration of the workflow of the experiments performed in example 1, wherein FE=flexion-extension, LB=lateral bending, AR=axial rotation, AS=anteroposterior shear, LS=lateral shear, ROM=range of motion;

FIG. 6 shows illustrations of the setup for biomechanical testing used in example 2 (FIG. 6A), illustration of the lateral view of the interspinous synthetic vertebropexy (FIG. 6B) and Illustration of the lateral view of the spinolaminar synthetic vertebropexy (FIG. 6C), as described in example 2;

FIG. 6A shows an illustration of the setup for biomechanical testing used in example 2;

FIG. 6B shows an illustration of the lateral view of the interspinous synthetic vertebropexy, as described in example 2;

FIG. 6C shows an illustration of the lateral view of the spinolaminar synthetic vertebropexy, as described in example 2;

FIG. 7 shows illustrations of interspinous vertebropexy after unilateral facetectomy using synthetic material;

FIG. 7A shows an illustration of interspinous vertebropexy in which the strap is passed through the holes to form a first loop, as described in example 2;

FIG. 7B shows an illustration of interspinous vertebropexy in which a second loop is made, as described in example 2;

FIG. 7C shows an illustration of interspinous vertebropexy in which the ends are brought together and tensioned, as described in example 2;

FIG. 8 shows illustrations of spinolaminar vertebropexy after unilateral facetectomy using synthetic material, as described in example 2;

FIG. 8A shows an illustration of spinolaminar vertebropexy in which the strap is passed behind the lamina of the cranial vertebral body and passes through the hole of the spinous process of the caudal vertebral body, as described in example 2;

FIG. 8B shows an illustration of spinolaminar vertebropexy in which the strap is guided under the lamina and brought back over the top, as described in example 2;

FIG. 8C shows an illustration of spinolaminar vertebropexy in which the other end of the strap is guided through the hole in the spinous process, as described in example 2;

FIG. 8D shows an illustration of spinolaminar vertebropexy in which both ends are brought together and tensioned, as described in example 2;

FIG. 8E shows an illustration of spinolaminar vertebropexy from a posterior view, as described in example 2;

FIG. 8F shows an illustration of spinolaminar vertebropexy from a lateral view, as described in example 2;

FIG. 9 shows the effect of microsurgical decompression, interspinous and spinolaminar fixation, and instrumentation; the y-axis shows the measured vertebral body segment range of motion (ROM) for different loading cases beyond the native state, as described in example 2;

FIG. 9A shows the effect of microsurgical decompression, interspinous and spinolaminar fixation, and instrumentation; the y-axis shows the measured vertebral body segment range of motion (ROM) for different loading cases beyond the native state, as described in example 2;

FIG. 9B shows the effect of microsurgical decompression, interspinous and spinolaminar fixation, and instrumentation; the y-axis shows the measured vertebral body segment range of motion (ROM) for different loading cases beyond the native state, as described in example 2;

FIG. 9C shows the effect of microsurgical decompression, interspinous and spinolaminar fixation, and instrumentation; the y-axis shows the measured vertebral body segment range of motion (ROM) for different loading cases beyond the native state, as described in example 2;

FIG. 9D shows the effect of microsurgical decompression, interspinous and spinolaminar fixation, and instrumentation; the y-axis shows the measured vertebral body segment range of motion (ROM) for different loading cases beyond the native state, as described in example 2;

FIG. 9E shows the effect of microsurgical decompression, interspinous and spinolaminar fixation, and instrumentation; the y-axis shows the measured vertebral body segment range of motion (ROM) for different loading cases beyond the native state, as described in example 2;

FIG. 10 shows comparisons of the effect of interspinous fixation using a fibercerclage and ligamentous interspinous fixation (vertebropexy); the y-axis shows the measured vertebral body segment range of motion (ROM) for different loading cases beyond the native state, as described in example 2;

FIG. 10A shows a comparison of the effect of interspinous fixation using a fibercerclage and ligamentous interspinous fixation (vertebropexy); the y-axis shows the measured vertebral body segment range of motion (ROM) for different loading cases beyond the native state, as described in example 2;

FIG. 10B shows a comparison of the effect of interspinous fixation using a fibercerclage and ligamentous interspinous fixation (vertebropexy); the y-axis shows the measured vertebral body segment range of motion (ROM) for different loading cases beyond the native state, as described in example 2;

FIG. 10C shows a comparison of the effect of interspinous fixation using a fibercerclage and ligamentous interspinous fixation (vertebropexy); the y-axis shows the measured vertebral body segment range of motion (ROM) for different loading cases beyond the native state, as described in example 2;

FIG. 10D shows a comparison of the effect of interspinous fixation using a fibercerclage and ligamentous interspinous fixation (vertebropexy); the y-axis shows the measured vertebral body segment range of motion (ROM) for different loading cases beyond the native state, as described in example 2;

FIG. 10E shows a comparison of the effect of interspinous fixation using a fibercerclage and ligamentous interspinous fixation (vertebropexy); the y-axis shows the measured vertebral body segment range of motion (ROM) for different loading cases beyond the native state, as described in example 2;

FIG. 11 shows the sample load deformation plots for each of the three graft types discussed in example 3 tested to failure;

FIG. 12 shows the sample load relaxation plot showing the difference in load between the initial load and the final load for each of the three graft types tested, as discussed in example 3;

FIG. 13 shows the sample creep plot showing the difference in displacement between the initial displacement and the final displacement for each of the 3 graft types tested, as discussed in example 3;

FIG. 14 illustrates the force to strain curve of an exemplary strap made of a synthetic material, as discussed in example 3;

FIG. 15 illustrates different embodiments of hybrid straps, which include a synthetic component and a natural component (FIG. 15A: the synthetic component and the natural component are arranged in a side-by-side arrangement; FIG. 15B: one of the components forms a sleeve around the other component);

FIG. 16 illustrates a selection of different looping variations that may be used for interspinous vertebropexy;

FIG. 16A shows a first and a second loop each passing through a cranial and a caudal hole in the cranial respectively the caudal spinous process;

FIG. 16B shows a first loop passing around the caudal edge of the caudal spinous process and the cranial edge of the cranial spinous process, while a second loop passes through a hole in the caudal spinous process and a hole in the cranial spinous process;

FIG. 16C shows a double-8-arrangement;

FIG. 16D shows a double-O-arrangement with both loops passing around the cranial edge of the cranial vertebra and the caudal edge of the caudal vertebra;

FIG. 16E shows both loops passing around the cranial edge of the cranial vertebra, but one loop passes around the caudal edge of the caudal vertebra, while other loop passes through hole in the caudal spinous process;

FIG. 17 illustrates a reinforcement structure to reinforce a hole in the spinous process;

FIG. 18 illustrates further variants in which the strap passes through a loop formed by an intermediate section of the strap;

FIG. 18A illustrates a strap passed through a loop formed by the intermediate section of the strap two times;

FIG. 18B illustrates two straps fastened to each other on their respective front sections as well as on their respective rear sections;

FIG. 18C illustrates a strap passed two times through a loop formed by the intermediate section of the strap;

FIG. 19 illustrates further variants in which a portion of the tension is shifted to another part of the spine through a tension distribution structure;

FIG. 20 illustrates two variants featuring positional securing elements to secure the position of the strap with respect to displacement in the ventral or dorsal direction;

FIG. 20A illustrates positional securing element interconnecting two intermediate sections of the strap;

FIG. 20B illustrates positional securing element additionally passed around the inferior articular process of the cranial vertebra; and

FIG. 21 illustrates a further variant in which front and rear fastening fibers are used to interconnect the front end of the strap and the rear end of the strap.

### DESCRIPTION OF PREFERRED EMBODIMENTS

Reference will now be made in detail to certain embodiments, examples of which are illustrated in the accompanying drawings, in which some, but not all features are shown. Indeed, embodiments disclosed herein may be embodied in many different forms and should not be construed as limited to the embodiments set forth herein; rather, these embodiments are provided so that this disclosure will satisfy applicable legal requirements. Whenever possible, like reference numbers will be used to refer to like components or parts.

To illustrate variants of the invention, two related studies were performed, which are outlined in detail below. In the first study (see example 1), fifteen spinal segments were biomechanically tested in a stepwise surgical decompression and ligamentous stabilization study. Stabilization was achieved with a gracilis or semitendinosus tendon allograft, which was attached to the spinous process (interspinous vertebropexy) or the laminae (interlaminar vertebropexy) in form of a loop. The specimens were tested (1) in the native state, after (2) microsurgical decompression, (3) interspinous vertebropexy, (4) midline decompression, and (5) interlaminar vertebropexy. In the intact state and after every surgical step, the segments were loaded in flexion-extension (FE), lateral shear (LS), lateral bending (LB), anterior shear (AS) and axial rotation (AR). In the second study (see example 2), twelve spinal segments (Th12/L1:4, L2/3:4, L4/5:4) were tested in a stepwise surgical decompression and stabilization study. Stabilization was achieved with a FiberTape cerclage, which was pulled through the spinous process (interspinous technique) or one spinous process and around both laminae (spinolaminar technique). The specimens were tested (1) in the native state, after (2) unilateral laminotomy, (3) interspinous vertebropexy and (4) spinolaminar vertebropexy. The segments were loaded in flexion-extension (FE), lateral shear (LS), lateral bending (LB), anterior shear (AS) and axial rotation (AR). In the following, the two examples are described in detail.

### 1. First Example

### 1.1 Materials and Methods of Example 1

### a) Dissection, Preparation and Storage

Fifteen spinal segments (TH12/L1:3, L1/2:3, L2/3:3, L3/4:3, L4/5:3) originating from seven fresh frozen cadavers (Table 1; Science Care, Phoenix, AZ, USA) were tested. After thawing CT scans (SOMATOM Edge Plus, Siemens Healthcare GmbH, Erlangen, Germany) were performed to exclude bony defects. The specimens were carefully dissected without harming bony processes, paraspinal ligaments or the intervertebral discs. After preparation, the segments were

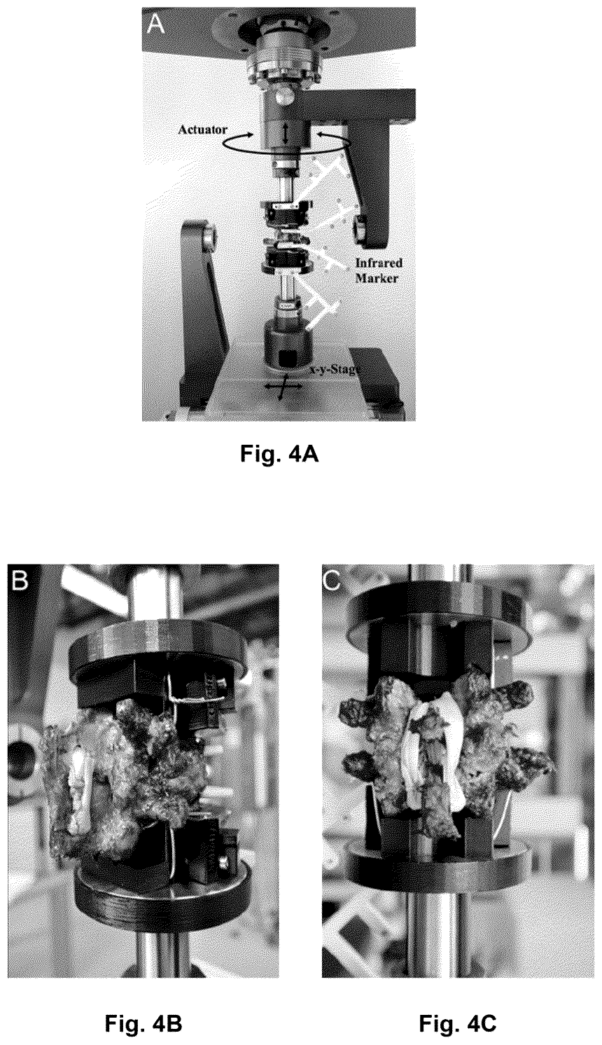

mounted on a testing machine (FIG. 4A) with individualized 3D-printed-clamps [12].

TABLE 1
Specimen information

| | | | | | Height | Weight | BMI |
|---|---|---|---|---|---|---|---|
| Specimen | Level | Sex | Age | Cause of death | (cm) | (kg) | (kg/m²) |
| C200862 | L1L2 | Male | 68 | Metastatic malignancy of the stomach | 177.8 | 55.3 | 17.5 |
| C200862 | L314 | Male | 68 | Metastatic malignancy of the stomach | 177.8 | 55.3 | 17.5 |
| S201932 | L1L2 | Female | 56 | Anoxic brain failure | 160 | 67.1 | 26.2 |
| S201932 | L3L4 | Female | 56 | Anoxic brain failure | 160 | 67.1 | 26.2 |
| S201942 | L1L2 | Male | 49 | Probable atherosclerotic coronary disease | 167.6 | 103.9 | 40 |
| S201942 | L3L4 | Male | 49 | Probable atherosclerotic coronary disease | 167.6 | 103.9 | 40 |
| S200838 | L4L5 | Male | 45 | Pending | 177.8 | 81.6 | 25.8 |
| S210555 | TH12L1 | Male | 57 | Acute cardiac arrest | 182.9 | 68.5 | 20.5 |
| S210555 | L2L3 | Male | 57 | Acute cardiac arrest | 182.9 | 68.5 | 20.5 |
| S210555 | L4L5 | Male | 57 | Acute cardiac arrest | 182.9 | 68.5 | 20.5 |
| L201826 | TH12L1 | Female | 62 | Acute respiratory failure | 167.6 | 132 | 47 |
| L201826 | L2L3 | Female | 62 | Acute respiratory failure | 167.6 | 132 | 47 |
| S210473 | TH12L1 | Male | 59 | Congestive heart failure | 167.6 | 66.2 | 23.6 |
| S210473 | L2L3 | Male | 59 | Congestive heart failure | 167.6 | 66.2 | 23.6 |
| S210473 | L4L5 | Male | 59 | Congestive heart failure | 167.6 | 66.2 | 23.6 |

### b) Description of the Stepwise Surgical Decompression and Techniques of Vertebropexy

### Microsurgical Decompression and Interspinous Vertebropexy:

A bilateral approach was used with sparing laminotomy of the overlying and underlying lamina. Then a flavectomy was performed from cranial to caudal followed by a recessotomy in a standard fashion.

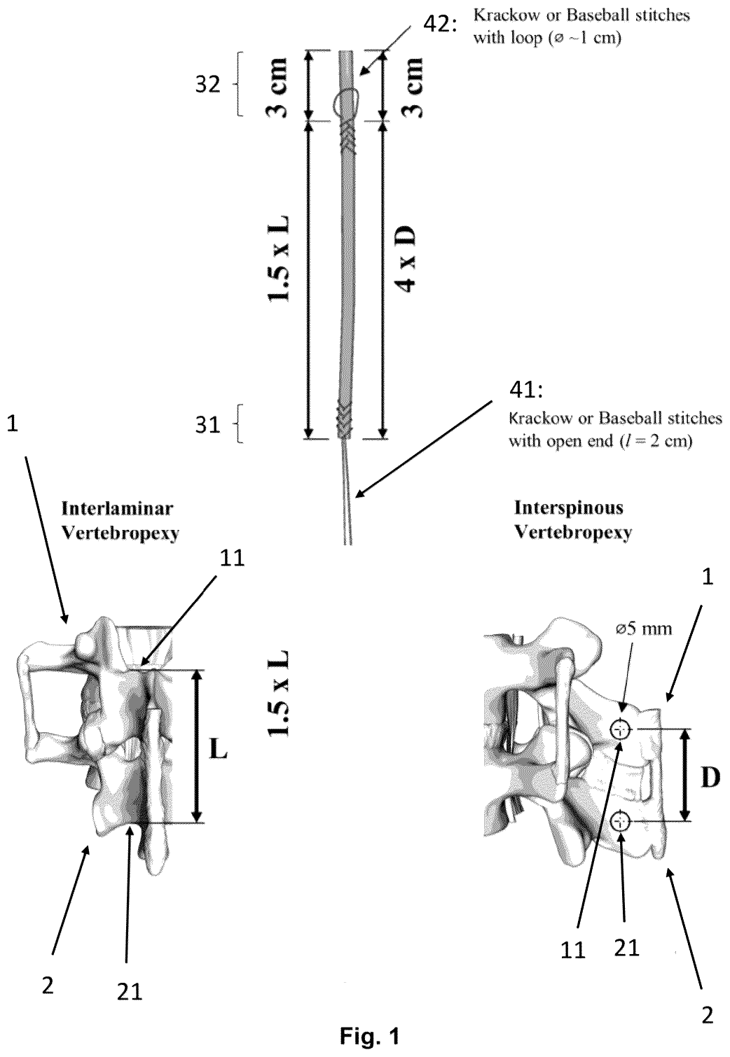

With reference to FIG. 1, both spinous processes were prepared for allograft passage by predrilling a 3.2-mm hole from one side to the other through the middle of the spinous process. The holes were overdrilled using a 5-mm drill bit, taking care not to create an iatrogenic fracture (cf. FIG. 1). A gracilis or semitendinosus tendon allograft (AlloSource, Centennial, Colorado) was prepared, thinning the allograft to a maximum diameter of 4 mm and reinforcing one end of the tendon with a Fiberwire No. 2 (Arthrex, Naples, Florida) using a 2-cm-long Krackow suture (FIG. 1; cf. Krackow K A, Cohn B T (1987) A new technique for passing tendon through bone. Brief note. J Bone Joint Surg Am Volume 69:922-4). For vertebropexy, the tendon graft was looped twice. The other end of the tendon was similarly reinforced with a Fiberwire No. 2, creating a loop in addition to the Krackow suture (FIG. 1).

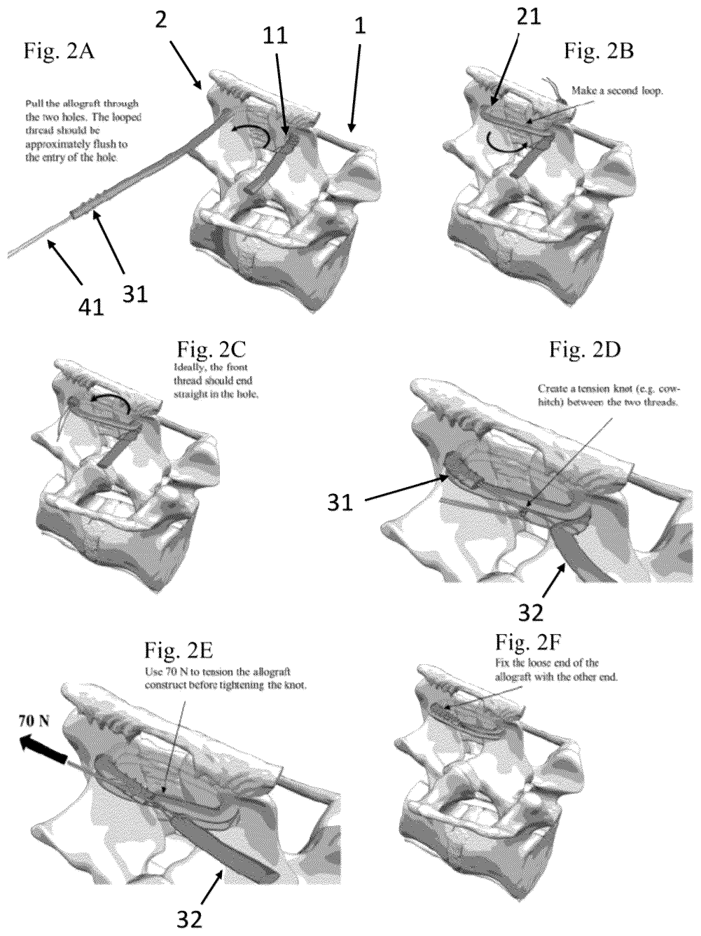

With reference to FIG. 2, thereafter, the allograft was pulled through the previously drilled holes in a double loop technique (FIG. 2A-C). An extension load of 5 Nm was applied via the static testing machine to simulate a prone position with physiological extension of the lumbar spine. The Fiberwire was knotted using the cow hitch technique (FIG. 2D): a double-stranded knot configuration with a loop on one side, secured by four half hitches. This technique is biomechanically stronger and stiffer compared to several other conventional knots (cf. Meyer D C, Bachmann E, Lädermann A, et al (2018) The best knot and suture configurations for high-strength suture material. An in vitro biomechanical study. Orthop Traumatol Surg Res 104:1277-1282). The knot was tightened with a force of 70N using a needle holder (FIG. 2E). The applied force was objectified with a force gauge. Finally, the second end of the tendon was sutured to the loop (FIG. 2F, FIG. 4B).

The same surgical approach is used for both steps (decompression and fixation) so no additional muscle attachments need to be released for fixation of the vertebral segment.

### Midline Decompression and Interlaminar Vertebropexy:

The supraspinous and interspinous ligaments were sharply removed with a Leksell rongeur, also the two spinous processes were partially removed. Midline decompression with the osteotome was performed, while care was taken not to harm the facet joints. The remaining ligamentum flavum was exposed and removed from cranial to caudal.

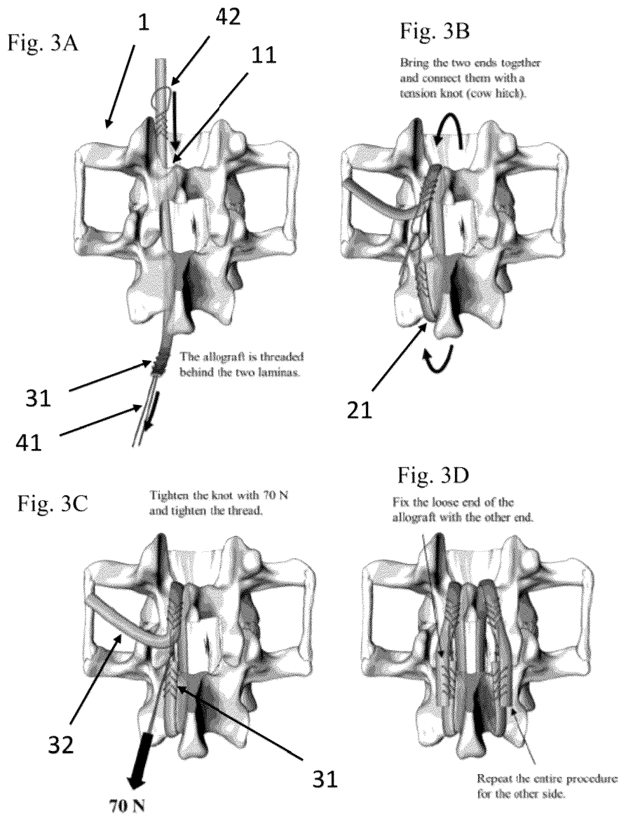

Two tendon allografts were reinforced in the same manner as described above (FIG. 1). With reference to FIG. 3, then the reinforced ends of the tendons were carefully passed under the laminae on both sides from cranial to caudal (FIG. 3A). A rongeur was used to pull the tips of both tendons up through the distal interlaminar window or above the inferior lamina (FIG. 3B). The segment was reloaded with 5 Nm extension and the Fiberwire was knotted bilaterally using a cow hitch and tightened with a force of 70N (FIG. 3C). The remaining part of the tendon was sutured to the loop (FIG. 3D, FIG. 4C).

### c) Biomechanical Experiments

With reference to FIG. 4, biomechanical testing of the 15 specimens was performed on a biaxial (linear & torsion) static testing machine (Zwick/Roell Allroundline 10 kN and testXpert III Software, ZwickRoell GmbH & Co. KG, Germany; FIG. 4A). The system is based on a traverse to generate vertical compression and tension and a torsion motor to generate torque in the horizontal plane. The machine was complemented with a testing setup consisting of an x-y-table and holding arms that allow for specimen fixation in a horizontal orientation for flexion-extension (FE), lateral shear (LS), lateral bending (LB), and anteroposterior shear (AS), and in a vertical orientation for axial rotation (AR). A customized mounting apparatus for the clamped specimens was used (cf. Cornaz F, Fasser M-R, Spirig J M, et al (2019) 3D Printed Clamps Improve Spine Specimen Fixation in Biomechanical Testing. J Biomech 98:109467), consisting of high-precision fitting rings, pins, and a mechanism to compress the connection with a defined load before tightening. Loading was applied to the cranial vertebra while the caudal vertebra was fixed to the x-y-table allowing for translational movement orthogonal to the loading direction. Coupled motions around the x- and y-axis were prevented, restricting all motions to the test plane. With this configuration, translation forces, as might occur with a fully constrained setup, are eliminated, resulting in pure moments and pure forces in the plane of interest. Further details on the test setup, including images of all loading configurations, are provided in a previously published study, which is incorporated herein by reference: Widmer J, Cornaz F, Scheibler G, et al (2020) Biomechanical contribution of spinal structures to stability of the lumbar spine-novel biomechanical insights. Spine J 20:1705-1716.

### d) Biomechanical Testing Protocol

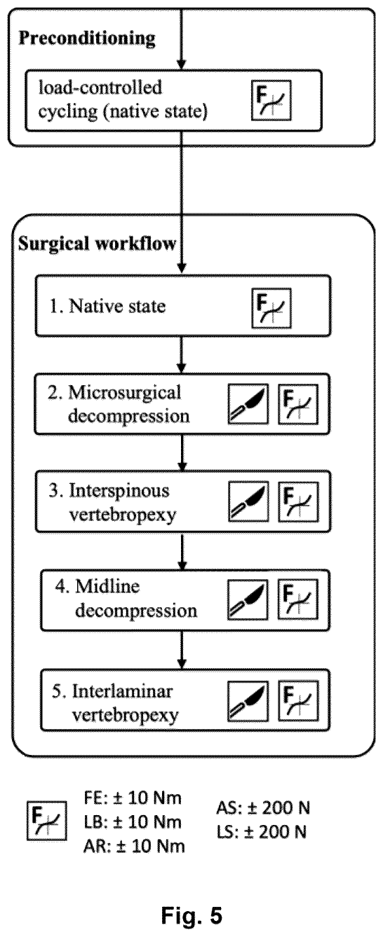

With reference to FIG. 5, each specimen was tested load-controlled (1) in the native state, after (2) microsurgical decompression, (3) interspinous vertebropexy, (4) midline decompression and (5) interlaminar vertebropexy. The surgical steps are illustrated in FIG. 5. After every surgical step, the segments were loaded in FE, LS, LB, AS and AR (in the listed order). For each loading case, 5 preloading cycles were conducted before the relative motion between the cranial and caudal vertebral bodies was recorded in the sixth cycle.

The segments were initially loaded with +10 Nm in the bending planes and +200 N in shear loading. In order to test the fixation techniques on extreme loads, slightly higher loads were chosen than the physiological range. Loading was applied with a velocity of 1°/sec in flexion-extension and lateral bending, 0.5°/sec in axial rotation, and 0.5 mm/sec in anterior, posterior and lateral shear (cf. Wilke H-J, Wenger K, Claes L (1998) Testing criteria for spinal implants: recommendations for the standardization of in vitro stability testing of spinal implants. Eur Spine J 7:148-154). During testing, specimens were kept moist by frequently spraying them with phosphate buffered saline.

### c) Data Analysis

The 3D motion data of the vertebrae (Atracsys Fusion Track 500, recording frequency 10 Hz, tracking accuracy 0.09 mm [RMS]) were used to correct the load-deflection curves of the testing machine. The centerline of the load-deflection hysteresis was fitted using a fifth-order polynomial. A standardized method (for further details, see the following publication, which is incorporated herein by reference: Widmer J, Cornaz F, Scheibler G, et al (2020) Biomechanical contribution of spinal structures to stability of the lumbar spine-novel biomechanical insights. Spine J 20:1705-1716) was used to separate positive/negative load directions in the load-deflection curves. For symmetrical load directions (LB, AR, and LS), the average values between negative and positive load (left, right) were used. Torsional preload in the sagittal plane was determined by analyzing the moment change in the neutral position between flexion and extension after each surgical step.

The statistical evaluation was performed with MATLAB (Matlab 2020b, Math Works, Massachusetts, USA). The difference in range of motion (ROM) relative to the native condition is reported with median and interquartile range. The Wilcoxon signed rank test was used for the statistical comparison of matched relative ROM values. Specifically, for the obtained results in each of the five loading directions, the ROM after the vertebropexies was compared with the movement after the respective previous decompression steps and a third comparison consists of the assessment of possible differences between the two vertebropexy steps. The mean values were used for segment-wise analysis, as only three data points were available per spinal level. Due to multiple comparisons, the significance level a was adjusted with Bonferroni corrections and set to be 0.05/2=0.025.

### 1.2 Results

The absolute ROM of the native segment and the segment after microsurgical decompression, interspinous vertebropexy, midline decompression, and interlaminar vertebropexy is shown in Table 2 according to loading case. The table shows the absolute mean range of motion native, after surgical decompression and stabilization by segment.

TABLE 2

| | | | | Midline | Interlaminar |
|---|---|---|---|---|---|
| | | Microsurgical | Verte- | decom- | verte- |
| | Native | decompression | bropexy | pression | bropexy |
| Th12/L1 | FE 5.7 | FE 5.9 | FE 2.2 | FE 6.4 | FE 2.7 |
| | LS 0.6 | LS 0.7 | LS 0.5 | LS 0.7 | LS 0.6 |
| | LB 5 | LB 5.2 | LB 5.2 | LB 5.6 | LB 5.5 |
| | AS 0.7 | AS 0.8 | AS 0.8 | AS 0.9 | AS 0.9 |
| | AR 1.5 | AR 1.5 | AR 1.5 | AR 1.8 | AR 1.8 |
| L1/L2 | FE 5.8 | FE 6.3 | FE 4 | FE 6.7 | FE 2.7 |
| | LS 1.8 | LS 2 | LS 1.9 | LS 2.1 | LS 1.8 |
| | LB 5.4 | LB 5.6 | LB 5 | LB 6 | LB 5.7 |
| | AS 2.3 | AS 2.7 | AS 2.5 | AS 2.9 | AS 2.6 |
| | AR 1.6 | AR 1.7 | AR 1.6 | AR 2.4 | AR 2.4 |
| L2/L3 | FE 9.2 | FE 9.6 | FE 2.7 | FE 10.6 | FE 4.3 |
| | LS 1.4 | LS 1.5 | LS 1 | LS 1.6 | LS 1.2 |
| | LB 10.1 | LB 10.4 | LB 9.6 | LB 11 | LB 9.8 |
| | AS 2.2 | AS 2.3 | AS 1.9 | AS 2.6 | AS 2.2 |
| | AR 3.7 | AR 3.8 | AR 3.1 | AR 3.9 | AR 3.3 |
| L3/L4 | FE 9.2 | FE 9.9 | FE 3.6 | FE 11.8 | FE 4.1 |
| | LS 3.6 | LS 3.7 | LS 3.3 | LS 4.6 | LS 3.9 |
| | LB 8.8 | LB 9.2 | LB 8 | LB 10.7 | LB 9.4 |
| | AS 3.6 | AS 3.9 | AS 3.5 | AS 5.2 | AS 4.5 |
| | AR 7.4 | AR 7.6 | AR 7 | AR 9.2 | AR 8.6 |
| L4/L5 | FE 12.2 | FE 12.6 | FE 4 | FE 13.1 | FE 4.7 |
| | LS 1.9 | LS 1.9 | LS 1.4 | LS 2 | LS 1.5 |
| | LB 9.3 | LB 9.7 | LB 9 | LB 10.1 | LB 9.3 |
| | AS 1.7 | AS 1.9 | AS 1.6 | AS 2 | AS 1.8 |
| | AR 3.7 | AR 3.8 | AR 3.3 | AR 3.8 | AR 3.6 |

for each of the five levels (TH12/L1, L1/2, L2/3, L3/4, L4/5) three cadaveric segments were available (15 segments in total); FE: flexion-extension (°); LS: lateral shear (mm); LB: lateral bending (°); AS: anteroposterior shear (mm); AR: axial rotation (°).

### a) Interspinous Vertebropexy

Interspinous vertebropexy significantly reduced the ROM in all loading scenarios. The ligamentous stabilization technique was able to decrease the ROM after microsurgical decompression in FE by almost 70% (p<0.001), in LS by 22% (p<0.001), in LB by 8% (p<0.001), in AS by 12% (p<0.01), and in AR by 9% (p<0.001). The effect of interspinous vertebropexy based on segments (see table 2 above) was relatively constant, and independent of the segment.

### b) Interlaminar Vertebropexy

Interlaminar vertebropexy significantly decreased the ROM of all segments compared to midline decompression in all loading scenarios. ROM was decreased by 70% (p<0.001) in FE, 18% (p<0.001) in LS, 11% (p<0.01) in LB, 7% (p<0.01) in AS, and 4% (p<0.01) in AR. The ROM was comparable between the segments.

### c) Comparison of the Two Vertebropexy Techniques

Vertebral segment ROM was significantly smaller with the interspinous vertebropexy compared to the interlaminar vertebropexy for all loading scenarios except FE. In FE, the effect of the two techniques was comparable (36.7% vs. 43.1%, p=0.08 (median; relative ROM after stabilization; native=100%). Significantly smaller vertebral segment ROM was achieved using interspinous vertebropexy in LS (81.7% vs. 98.1%; p<0.01), LB (95.9% vs. 100.3%; p<0.001), AS (96.3% vs. 115.9%; p<0.001), and AR (93.5% vs. 115.5%; p<0.001).

Overall, both techniques decreased vertebral body segment ROM in FE, LS and LB beyond the native state. The vertebropexy mainly reduced ROM in FE, in the other loading cases the effect was considerably smaller. The decompression steps led to increased ROM in each loading scenario compared to the native state.

### 2. Second Example

### 2.1 Materials and Methods of Example 2

### a) Dissection, Preparation and Storage

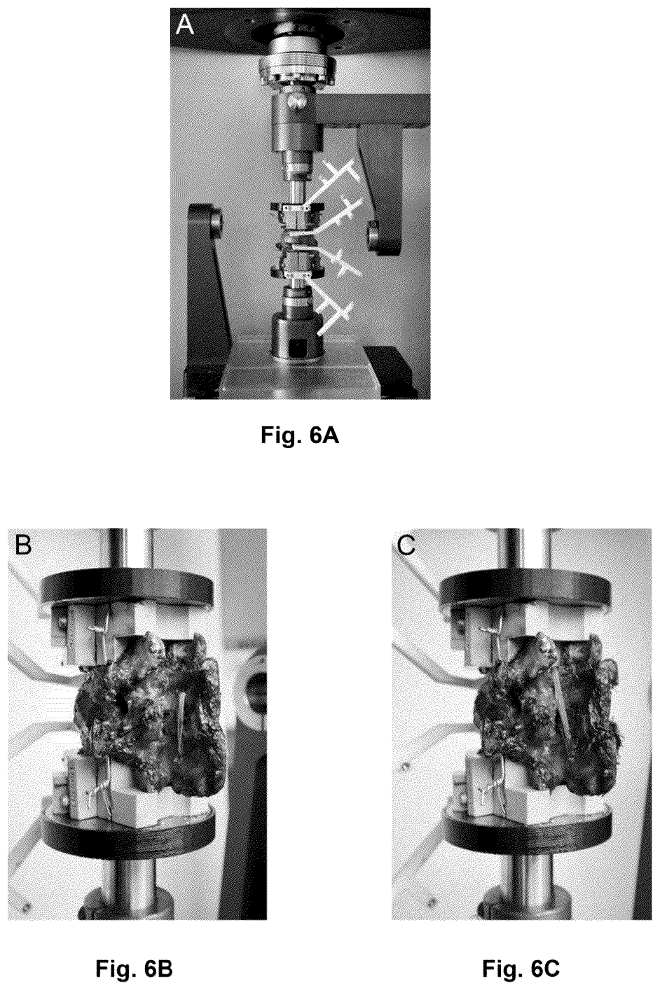

Twelve spinal segments (Th12/L1:4, L2/3:4, L4/5:4) originating from five fresh frozen cadavers (Table 3 below; Science Care, Phoenix, AZ, USA) were tested. Except for age-appropriate changes, the specimens were free of any osseous defects or deformities based on computed tomography scans (SOMATOM Edge Plus, Siemens Healthcare GmbH, Erlangen, Germany). After thawing, the cadavers were each separated into the vertebral segments Th12-L1, L2-L3, and L4-L5. The specimens were denuded of the surrounding muscle and connective tissue without harming the intersegmental ligamentous structures, facet joints, or intervertebral discs. After preparation, the segments were mounted on a testing machine (FIG. 6) with individualized 3D-printed-clamps (cf. Cornaz F, Fasser M-R, Spirig J M, et al (2019) 3D Printed Clamps Improve Spine Specimen Fixation in Biomechanical Testing, J Biomech 98:109467).

TABLE 3
Specimen information
Table 1 Specimen information

| | | | | | Height | Weight | BMI |
|---|---|---|---|---|---|---|---|
| Specimen | Level | Sex | Age | Cause of death | (cm) | (kg) | (kg/m²) |
| C220688 | TH12L1 | Male | 75 | Acute hypoxic and hypercapnic respiratory failure | 180.3 | 68 | 20.9 |
| C220688 | L2L3 | Male | 75 | Acute hypoxic and hypercapnic respiratory failure | 180.3 | 68 | 20.9 |
| C220707 | L4L5 | Female | 94 | COPD | 154.9 | 35.3 | 14.7 |
| L200232 | TH12L1 | Male | 71 | Cardiorespiratory arrest | 167.6 | 150.1 | 53.4 |
| L200232 | L2L3 | Male | 71 | Cardiorespiratory arrest | 167.6 | 150.1 | 53.4 |
| L200232 | L4L5 | Male | 71 | Cardiorespiratory arrest | 167.6 | 150.1 | 53.4 |
| L211459 | TH12L1 | Female | 78 | COPD, tobacco use | 170.2 | 93.4 | 32.3 |
| L211459 | L2L3 | Female | 78 | COPD, tobacco use | 170.2 | 93.4 | 32.3 |
| L211459 | L4L5 | Female | 78 | COPD, tobacco use | 170.2 | 93.4 | 32.3 |
| P220110 | TH12L1 | Female | 43 | Metastatic rectal adenocarcinoma | 160 | 68.9 | 26.9 |
| P220110 | L2L3 | Female | 43 | Metastatic rectal adenocarcinoma | 160 | 68.9 | 26.9 |
| P220110 | L4L5 | Female | 43 | Metastatic rectal adenocarcinoma | 160 | 68.9 | 26.9 |

### b) Description of the Stepwise Surgical Decompression and Techniques of the Synthetic Vertebropexies

Microsurgical Decompression with Unilateral Laminotomy and Interspinous Synthetic Vertebropexy:

A unilateral approach was used with sparing laminotomy of the overlying and underlying lamina. Then a flavectomy was performed from cranial to caudal followed by a recessotomy in a standard fashion.

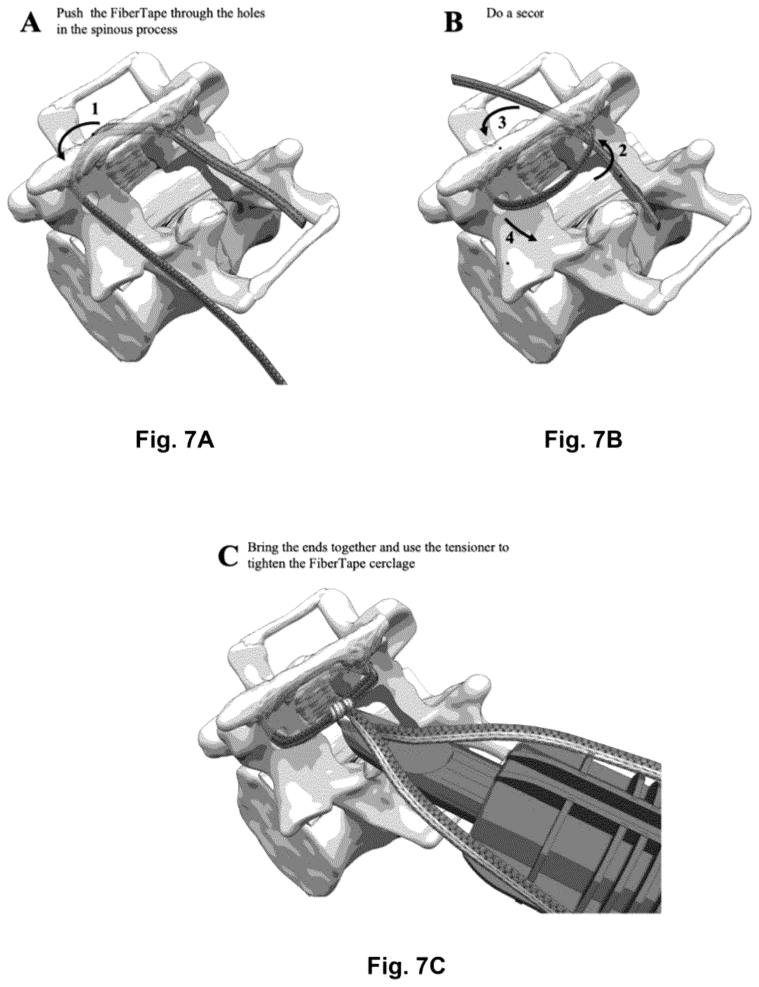

With reference to FIG. 7, for interspinous fixation, the technique of interspinous vertebropexy was followed, with the exception that synthetic material was used in the present biomechanical tests. Both spinous processes were prepared by drilling a 3.2-mm hole from one side to the other through the middle of the spinous process (FIG. 7). A FiberTape Cerclage (Arthrex, Naples, Florida) was pulled through the previously drilled holes in a double loop technique (FIG. 7). An extension load of 5 Nm was applied via the static testing machine to simulate a prone position with physiological extension of the lumbar spine. The cerclage was then tightened in a standardized manner with the corresponding tensioner, applying a force of approximately 40 pounds in each case (corresponds to the second marking on the tensioner). Afterwards, the cerclage was secured with five knots, using the tensioner to tighten the first knot.

### Spinolaminar Synthetic Vertebropexy:

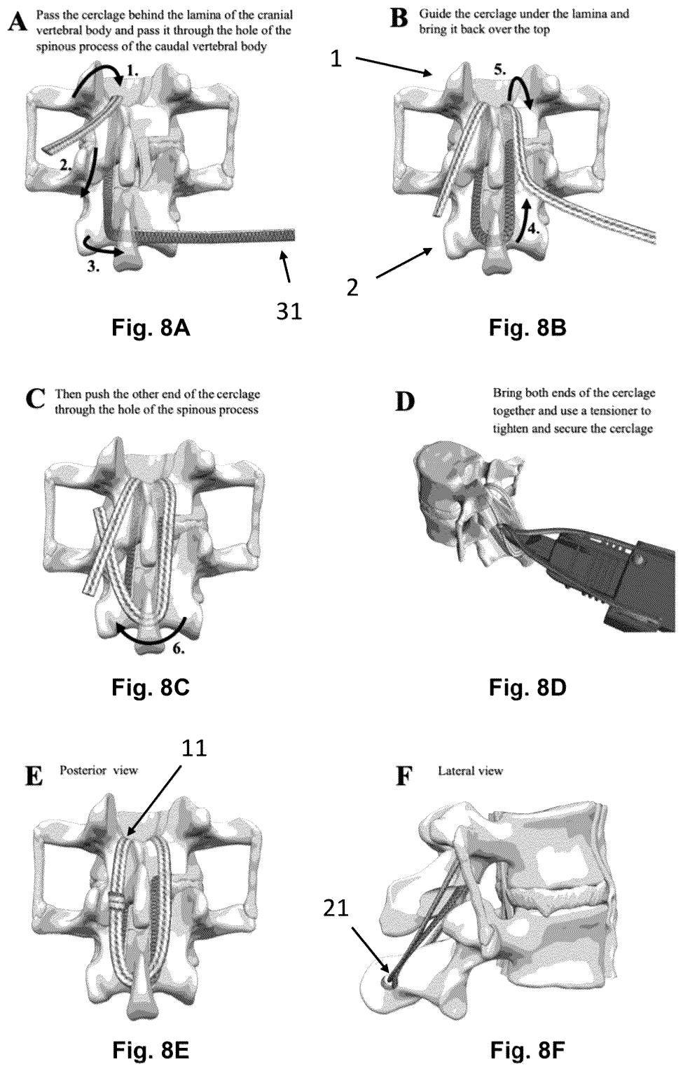

With reference to FIG. 8, a FiberTape cerclage was first passed through the pre-existing hole in the spinous process of the distal vertebra and then passed cranially anterior of the lamina of the proximal. The cerclage was then looped around the lamina and passed again through the hole in the spinous process of the distal vertebra. The same procedure was followed on the opposite side of the vertebra (FIG. 8). The FiberTape cerclage was then tightened as described above and secured with five knots.

### c) Biomechanical Experiments

Biomechanical testing of the twelve specimens was performed on a biaxial (linear and torsion) static testing machine (Zwick/Roell Allroundline 10 kN and testXpert III Software, ZwickRoell GmbH & Co. KG, Germany; FIG. 1). The system is based on a traverse: vertical compression and tension can be generated, and torque can be generated in the horizontal plane using a torsion motor. The machine was complemented with a test setup. It consisted of an x-y table and holding arms, allowing specimens to be fixed in horizontal orientation for flexion-extension (FE), lateral shear (LS), lateral bending (LB) and anteroposterior shear (AS), and in vertical orientation for axial rotation (AR). A customized mounting jig was used for the clamped specimens (cf. Cornaz F, Fasser M-R, Spirig J M, et al (2019) 3D Printed Clamps Improve Spine Specimen Fixation in Biomechanical Testing. J Biomech 98:109467). In each case, the cranial vertebra was loaded while the caudal vertebra was fixed on the x-y table. This allowed translational motion orthogonal to the loading direction, generating pure bending moments and shear forces. The setup allowed specimen fixation with extremely high reproducibility (variability) <0.005° (cf. Widmer J, Cornaz F, Scheibler G, et al (2020) Biomechanical contribution of spinal structures to stability of the lumbar spine-novel biomechanical insights, Spine J 20:1705-1716).

### d) Biomechanical Testing Protocol

Each specimen was tested load-controlled (1) in the native state, after (2) unilateral laminotomy, (3) interspinous vertebropexy and (4) spinolaminar vertebropexy. After each surgical step, the segments were loaded in FE, LS, LB, AS, and AR (in the order listed). For each loading case, five preloading cycles were performed before recording the relative motion between the cranial and caudal vertebral bodies in the sixth cycle. Data were recorded throughout the loading cycle, and the amplitude of translational motion of the markers (LS, AS) and projected angulation in the plane of motion (FE, LB, AR) were evaluated.

The segments were initially loaded with ±10 Nm in the bending planes and ±200 N in the shear loading. Slightly higher loads than in the physiological range were chosen to test the fixation techniques at extreme loading. Loading was applied at a rate of 1°/sec for flexion-extension and lateral bending, 0.5°/sec for axial rotation, and 0.5 mm/sec for anterior, posterior, and lateral shear (cf. Cornaz F, Widmer J, Farshad-Amacker N A, et al (2020) Biomechanical Contributions of Spinal Structures with Different Degrees of Disc Degeneration, Spine 46: E869-E877). During testing, specimens were kept moist by frequent spraying with phosphate-buffered saline.

The following comparisons of segmental ROM were undertaken: (1) microsurgical decompression with unilateral laminotomy versus synthetic vertebropexies, (2) synthetic interspinous versus spinolaminar vertebropexy, (3) ligamentous interspinous vertebropexy versus synthetic interspinous vertebropexy, and (4) synthetic vertebropexies versus dorsal fusion. For this purpose, data sets from the previously published studies were used, which are incorporated herein by reference: Farshad M, Burkhard M D, Spirig J M (2021) Occipitopexy as a Fusionless Solution for Dropped Head Syndrome: A Case Report. JBJS Case Connect 11 (3): e21.00049.

### c) Data Analysis

The statistical evaluation was performed with MATLAB (Matlab 2020b, Math Works, Massachusetts, USA). The difference in range of motion (ROM) relative to the native condition is reported with the median and interquartile range. The Wilcoxon signed rank test was used for the statistical comparison of matched relative ROM values. Specifically, for the obtained results in each of the five loading directions, the relative ROM after the synthetic vertebropexies was compared with the movement after microsurgical decompression with unilateral laminotomy and a third comparison consisted of the assessment of possible differences between the two synthetic vertebropexies. Unpaired comparisons of the relative ROM after the synthetic vertebropexies and measurements of the same parameter after microsurgical decompression and instrumentation were performed with Wilcoxon rank sum tests. Furthermore, interspinous synthetic vertebropexy was compared with the ROM after interspinous ligamentous vertebropexy. The significance level alpha was set to 0.05 and the p-values were corrected according to Bonferroni to adjust for multiple comparisons.

### 1.2 Results

a) Effect of Synthetic Vertebropexies after Microsurgical Decompression with Unilateral Laminotomy

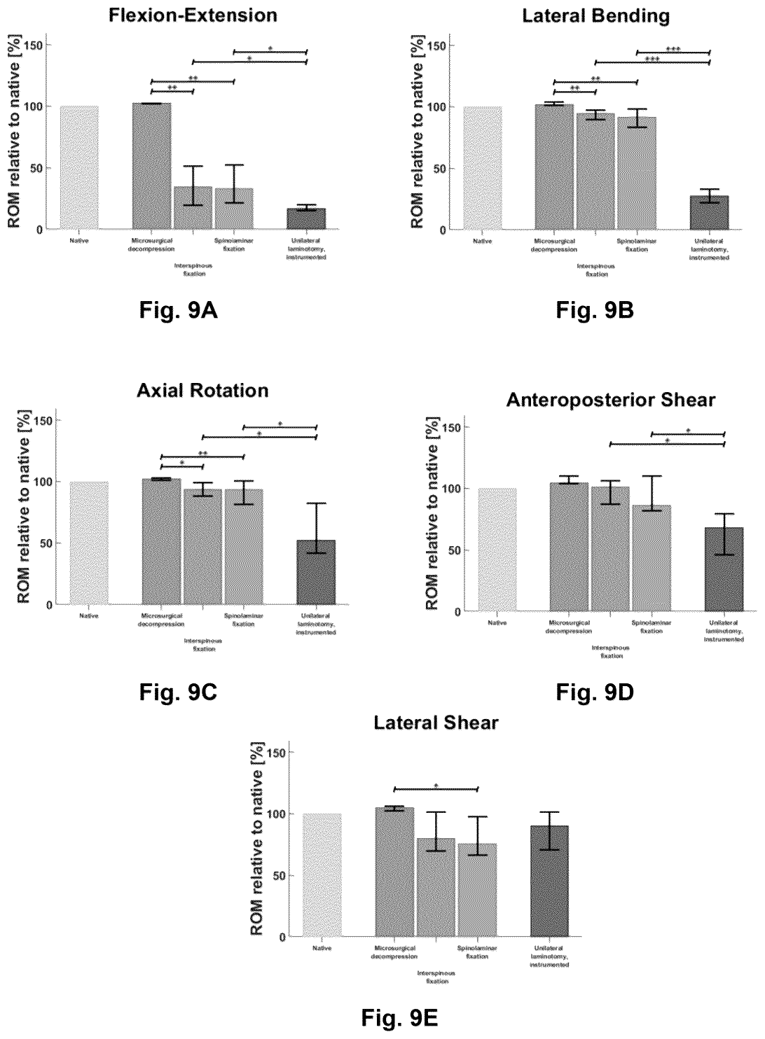

With reference to FIG. 9, microsurgical decompression increased native ROM in all loading cases (FIG. 9): in FE by 2%, in LS by 5%, in LB by 1%, in AS by 4%, and in AR by 2%.

Interspinous fixation significantly reduced ROM after microsurgical decompression in FE by 66% (p=0.003), in LB by 7% (p=0.006), and in AR by 9% (p=0.02). Shear movements (LS and AS) were also reduced, although not significantly: in LS reduction by 24% (p=0.07), in AS reduction by 3% (p=0.21).

Spinolaminar fixation significantly reduced ROM after microsurgical decompression in FE by 68% (p=0.003), in LS by 28% (p=0.01), in LB by 10% (p=0.003), and AR by 8% (p=0.003). AS was also reduced, although not significantly: reduction by 18% (p=0.06).

### b) Comparison of Interspinous and Spinolaminar Vertebropexy Using Synthetic Material

With reference to FIG. 9, the effect of the two techniques was comparable: FE 34.6% vs. 32.9%, p=1 (median; relative ROM after interspinous versus spinolaminar fixation; native=100%); LS 79.9% vs. 75.2%, p=1; LB 94% vs. 91.2%, p=1; AS 100.8% vs. 86%, p=1; and AR 93.1% vs. 93.3%, p=1.

Overall, both techniques decreased vertebral body segment ROM in all loading cases beyond the native state, except for the interspinous fixation technique, which only slightly influenced AS movement.

The spinolaminar technique had a higher effect on shear motion compared to interspinous fixation. Overall, both techniques mainly influenced ROM in FE.

### c) Comparison of Ligamentous Interspinous Fixation (Ligamentous Vertebropexy) and Interspinous Fixation Using a Fibercerclage (Synthetic Vertebropexy)

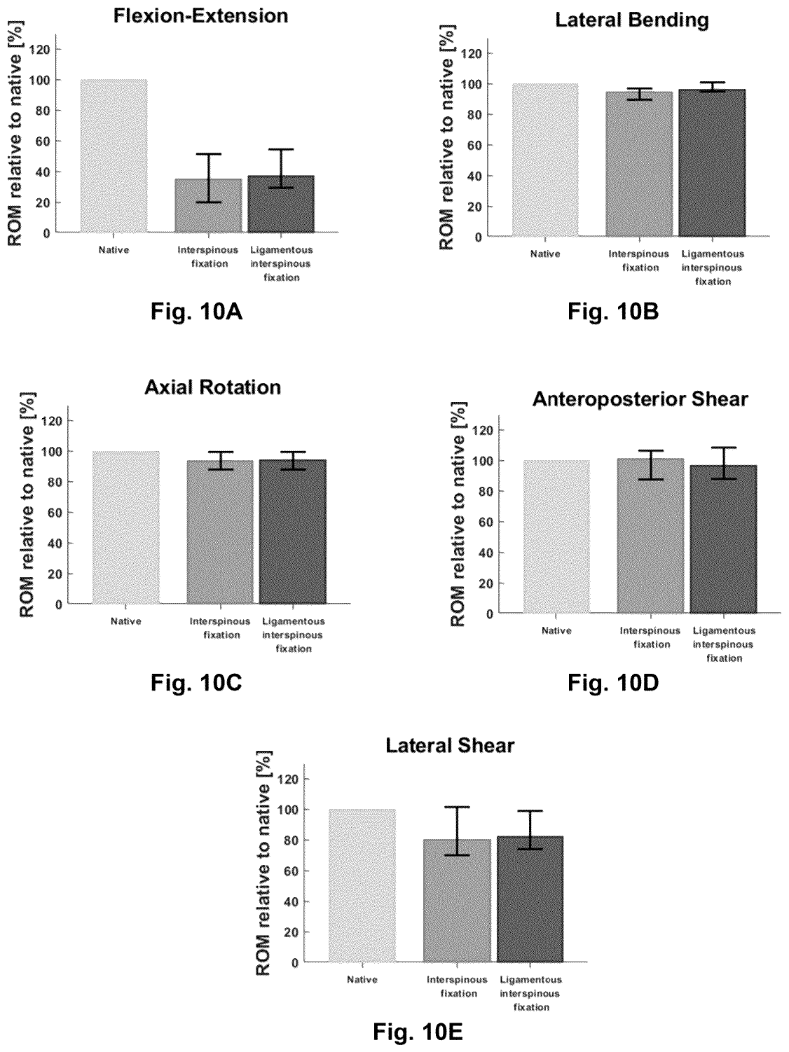

With reference to FIG. 10, the effect of the two techniques was comparable and thus largely independent of the material used for stabilization: FE 34.6% vs. 36.8%, p=1 (median; relative ROM after interspinous versus ligamentous interspinous fixation; native=100%); LS 79.9% vs. 81.7%, p=1; LB 94% vs. 95.9%, p=0.9; AS 100.8% vs. 96.3%, p=1; and AR 93.1% vs. 93.5%, p=1.

### d) Comparison of Synthetic Vertebropexies and Dorsal Fusion

With reference to FIG. 9, both synthetic vertebropexies affected all loading cases, but significantly less than fusion by connecting the inserted pedicle screws (FIG. 9). After fusion, all loading cases, except LS (LS 14% vs. 24% (p=1) vs. 28% (p=1)), showed significantly higher median relative reductions compared to interspinous and spinolaminar synthetic vertebropexy: FE 83.3% vs. 66% (median; relative reduction after fusion versus interspinous fixation, p=0.026) vs. 68% (relative reduction after fusion versus spinolaminar fixation, p=0.04); LB 73.3% vs. 7% (p<0.001) vs. 10% (p<0.001); AS 34.9% vs. 3% (p=0.02) vs. 18% (p=0.02); and AR 49% vs. 9% (p=0.02) vs. 8% (p=0.02).

### 3. Third Example

Example 3 describes the mechanical properties of some variants of the strap, as well as possible measurements to measure these mechanical properties. Only exemplary variants of the straps are discussed in example 3. Other variants of the strap may also be envisioned.

It should be noted that as outlined in the following, the third example is generally focused on double-looped semitendinosus and gracilis (DLSTG) graft, which was notably formed by looping the graft around the two vertebrae two times, thereby forming a double-O-structure. As the skilled person knows, some of the mechanical properties depend on the total number of loops. For example, if a tensile strength between the adjacent vertebrae is measured, the tensile strength will increase with an increase in the number of loops. Thus, it is understood that if the mechanical measurements discussed in the context of DLSTG in the third example are to be translated to the graft itself, a factor of 1/(2n) needs to be applied, where n is the total number of loops. For example, if the ultimate load of DLSTG is determined to be 2,913 N, then the ultimate load of the respective graft would be a quarter thereof, namely 728.25 N. It is understood that the applied factor of ¼ takes into account the fact that in DLSTG, each of the two loops includes two opposite sides contributing to the load.

### 3.1 Part 1: Grafts

In some variants looped semitendinosus and gracilis (DLSTG) graft may be used. Depending on the application, a combination of semitendinosus and gracilis may be used in the form of a double loop, e.g. for interspinous vertebropexy, or the combination may be used in the form of two single loops, e.g. for interlaminar vertebropexy.

Part 1 or example 3 describes selected mechanical properties of exemplary grafts that may be used for the strap. Specifically, part 1 discusses anterior tibialis tendons, posterior tibialis tendons and a double-looped semitendinosus and gracilis (DLSTG) graft.

### Ramp-to-Failure Test

Double-loop grafts were clamped 95 mm from the steel bar with the liquid nitrogen freeze clamp at room temperature. The grafts were preconditioned between 20 N and 250 N by applying 10 cycles at 0.1 Hz; thereafter, a load of 20 N was maintained to set the initial gauge length until testing (note: this means that the origin of the curve at which displacement/strain=0 was placed at 20 N load). The load-to-failure test was performed 15 minutes after preconditioning by pulling the graft to failure at a strain rate of 2%/s (1.5 mm/s).

Table 4 below shows a comparison of the structural properties between the anterior tibialis and DLSTG graft and the posterior tibialis and DLSTG graft for 95-mm graft length (mean±SD).

TABLE 4

| | Ultimate Load | Linear Stiffness | Ultimate | |
|---|---|---|---|---|
| Type of Graft | (N) | (N/mm) | Displacement (mm) | Area (mm²) |
| Anterior | 4,122 ± 893* | 460 ± 101 | 12.0 ± 3.0* | 48.2 ± 11.8 |
| Tibialis | (P = .005) | (NS, P = .283) | (P = .007) | (NS, P = .432) |
| Posterior | 3,594 ± 1,330 | 379 ± 143 | 12.5 ± 2.3* | 41.9 ± 17.3 |
| Tibialis | (NS, P = .204) | (NS, P = .467) | (P < .001) | (NS, P = .695) |
| DLSTG | 2,913 ± 645 | 418 ± 36 | 8.4 ± 1.3 | 44.4 ± 6.7 |

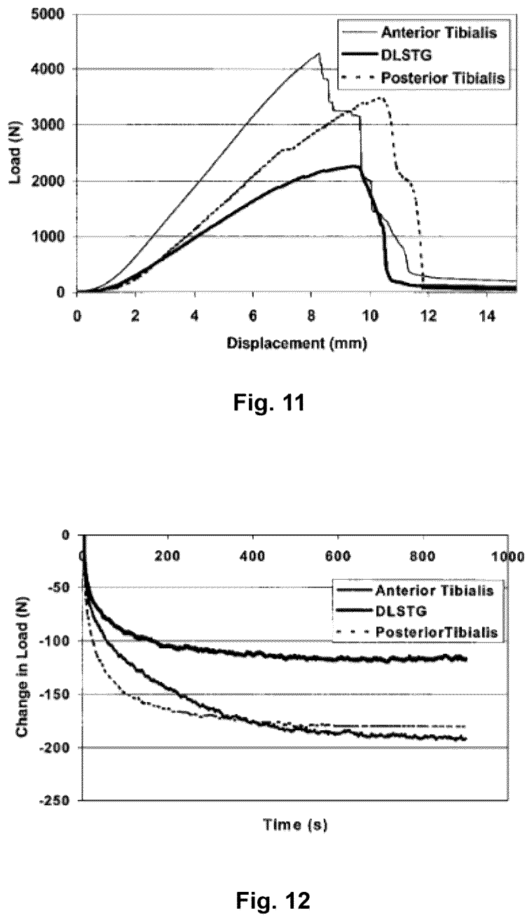

FIG. 11 shows the sample load deformation plots for each of the three graft types tested to failure. Stiffness and tensile modulus were determined in the linear region from 50% to 75% of the ultimate load. The ultimate load and stiffness of both single-loop tibialis tendon grafts were either similar to or greater than that of the DLSTG graft.

### Stress Relaxation Test

In a further test, double-loop grafts were clamped 75 mm from the steel bar with the liquid nitrogen freeze clamp. The test was performed 15 minutes after preconditioning. The test measured the decrease in load under a constant displacement (i.e., stress relaxation test) and was conducted by elongating the graft to, 2.5% strain at a rate of 250 mm/s. The load was recorded at 4 Hz while the displacement was held constant for either 15 minutes or until the load remained unchanged over 1 minute (i.e., less than 0.1% decrease in load).

FIG. 12 shows the sample load relaxation plot showing the difference in load between the initial load and the final load for each of the three graft types tested. Only the single-loop anterior tibialis tendon graft relaxed more than the DLSTG graft.

### Creep Test

In a further test, the graft was refrigerated overnight, and 24 hours later it was equilibrated to room temperature and preconditioned. The test was performed 15 minutes after preconditioning and measured the increase in displacement under a constant load (i.e., creep test) and was conducted by applying a 20 N load and increasing the load to 250 N at a rate of 315 N/s. The displacement was recorded at 1 Hz while the load was held constant at 250 N for either 15 minutes or until the displacement remained unchanged over 1 minute (i.e., less than a 0.1% increase in displacement.

FIG. 13 shows the sample creep plot showing the difference in displacement between the initial displacement and the final displacement for each of the 3 graft types tested. Both single-loop tibialis tendon grafts crept more than the DLSTG graft, but the difference is not considered clinically important.

Table 5 shows a comparison of material properties between the anterior tibialis and DLSTG graft and the posterior tibialis and DLSTG graft (mean±SD).

TABLE 5

| Type of | Modulus | Ultimate Stress | Ultimate Strain |
|---|---|---|---|
| Graft | (MPa) | (MPa) | (%) |
| Anterior | 847 ± 301 | 89.8 ± 19.4* | 12.7 ± 3.2* |
| Tibialis | (NS, P = .618) | (P = .007) | (P = .006) |
| Posterior | 905 ± 230 | 89.1 ± 15.4* | 13.2 ± 2.4* |
| Tibialis | (NS, P = .983) | (P = .003) | (P < .001) |
| DLSTG | 904 ± 99 | 65.6 ± 12.0 | 8.8 ± 1.4 |

Abbreviation: NS, not significant.
*Denotes property significantly different from that of DLSTG graft.

Table 6 shows a comparison of viscoelastic properties between the anterior tibialis and DLSTG graft and the posterior tibialis and DLSTG graft for 75 mm graft length. Thus, depending on the application, the strap may have a decrease in load under a constant displacement from 100 N to 250 N, when measured with a strap length of 75 mm. Additionally or alternatively, the strap may have an increase in displacement under a constant load from 0.1 mm to 0.6 mm, when measured with a strap length of 75 mm.

TABLE 6

| | Decrease in Load | Increase in |
|---|---|---|
| Type of | Under a Constant | Displacement Under |
| Graft | Displacement (N) | a Constant Load (mm) |
| Anterior | 215 ± 92* | 0.3 ± 0.1* |
| Tibialis | (P = .027) | (P = .004) |
| Posterior | 197 ± 91 | 0.4 ± 0.1* |
| Tibistis | (NS, P = .066) | (P < .001) |
| DLSTG | 134 ± 38 | 0.2 ± 0.0 |

Abbreviation: NS, not significant.
*Denotes property significantly different from that of DLSTG graft.

### 3.2 Part 2: Synthetic Material

In some variants, the strap may comprise or consist of a synthetic material. Depending on the application, the strap may have an elliptical cross-section. The cross-section may, for example, have a length from 8 mm to 12 mm and a height from 1 mm to 5 mm in a relaxed state.

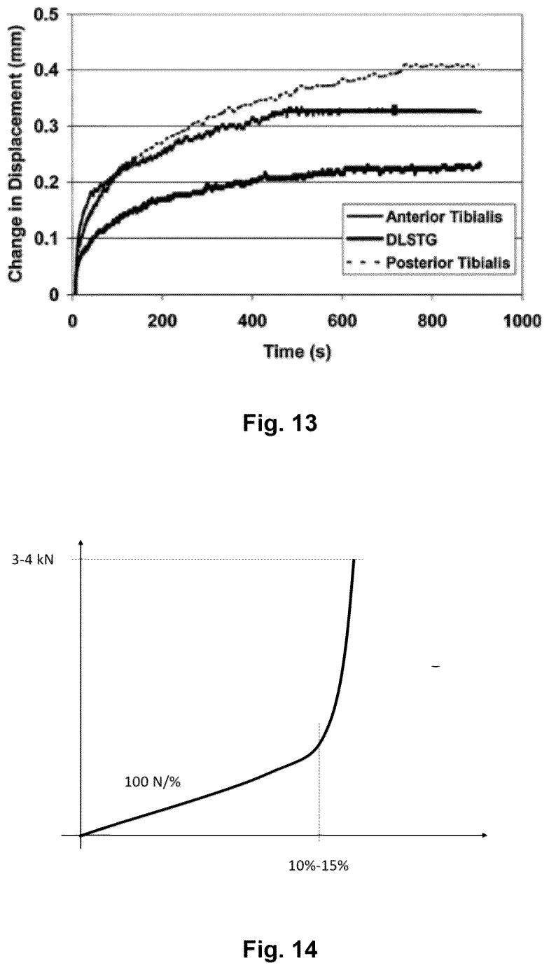

FIG. 14 illustrates, for a diameter from 2 mm to 3 mm, the force to strain curve of an exemplary strap made of a synthetic material. A first section of the curve can be described as a linear response (Force to strain) with an inclination from 40 to 100 N per % strain. As an example, the inclination in the first section may be from 300 to 550 N/mm strain, preferably from 400 to 450 N/mm strain. In a second section of the curve, starting at a strain between 8 and 15% (yield point), the curve is steeper with an inclination from 500 to 1000 N per % strain. The ultimate tensile strength (failure force) may, for example, be from 3000 N to 4000 N. The ultimate tensile strain (displacement/strain of breaking point) may, for example, be from 17% to 20%. The % refers to the initial length of the ligament, possibly at preload 70N (force at which strain/displacement is set to 0).

### 4. Fourth Example

Example 4 describes selected further variants of the method and of the strap.

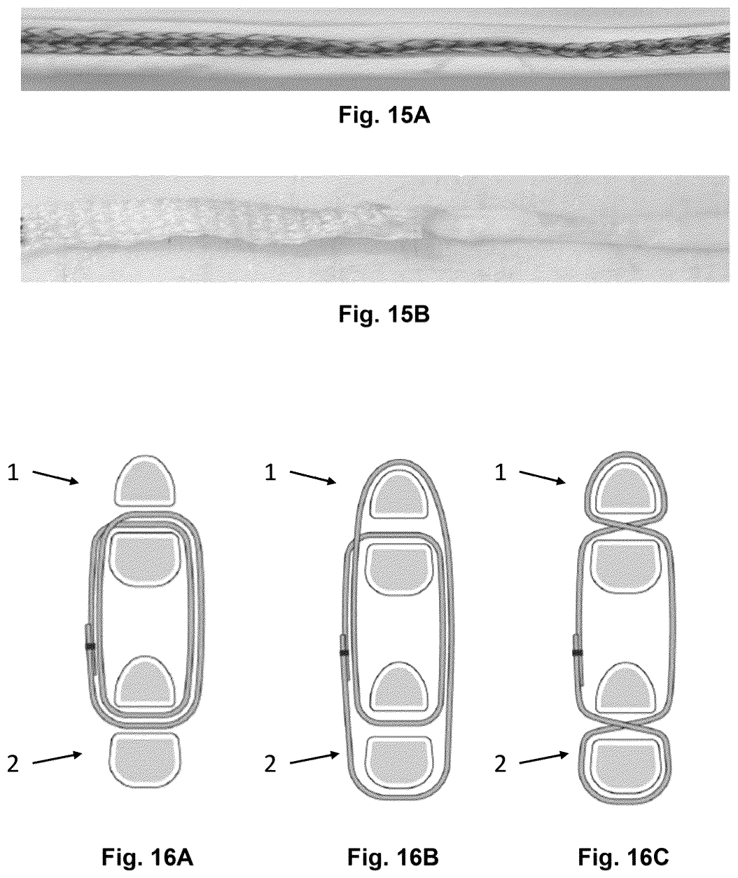

FIG. 15 illustrates different embodiments of hybrid straps, which include a synthetic component and a natural component (e.g. ligament or tendon, such as autograft, allograft or xenograft). The synthetic component may e.g. be a textile, such as a braided textile. As illustrated in FIG. 15A, the synthetic component and the natural component may be arranged adjacent to each other, wherein the synthetic component is arranged on a first lateral surface of the ligament component. As illustrated in FIG. 15B, in a further variant, one of the components may form a sleeve circumferentially encompassing at least a longitudinal section of the other component. For example, the synthetic component may form a sleeve (e.g. having a hollow cylindrical shape) encompassing a section of the ligament.

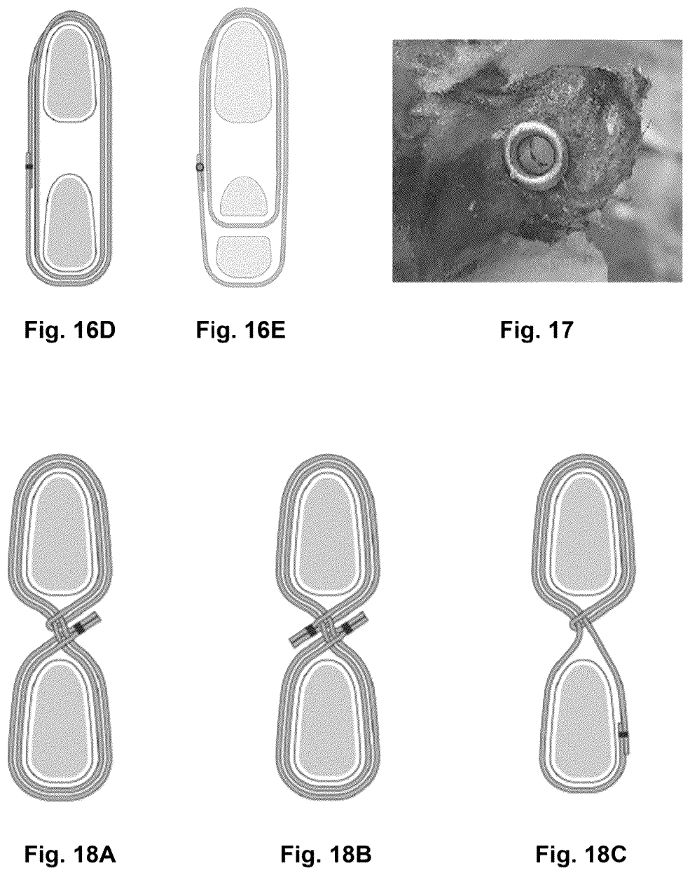

FIG. 16 illustrates a selection of different looping variations that may be used for interspinous vertebropexy. In the illustrated variants, a double loop is formed to interconnect a cranial vertebra 1 and a caudal vertebra 2. However, it is noted that the number of loops may also be changed, for example a single loop, a triple loop or even further loops may also be used. In the embodiments illustrated in FIG. 16, the cranial vertebra 1 and the caudal vertebra 2 are shown from a dorsal view. More specifically, a dorsal view of the spinous process of the cranial vertebra 1 and of the spinous process of the caudal vertebra 2 is shown. Depending on the application, one or both of the spinous processes may include a hole, which is also illustrated in the figures. For example, in FIGS. 16A, 16B and 16C, both spinous processes each comprise a hole, which is illustrated in the respective figures. As discussed in further detail below, the hole may be used to pass the strap through the hole.

In FIG. 16A, the strap forms a double loop through the respective holes in the spinous processes of the cranial and caudal vertebrae. The two loops contact each other and are arranged adjacent to each other.

In FIG. 16B, the strap also forms a double loop, but the double loop comprise a first loop that is formed using different fastening edges than the second loop. Specifically, a first loop is formed by passing the strap around a caudal edge of the spinous process of the caudal vertebra, followed by guiding the strap towards the cranial vertebra, then passing the strap around a cranial edge of the spinous process of the cranial vertebra, and then guiding the strap back towards the caudal vertebra, thereby defining a first loop. The second loop is then formed by passing the strap through a hole in the spinous process of the caudal vertebra, then guiding the strap back towards the cranial vertebra, then passing the strap through a hole in the spinous process of the cranial vertebra, and then guiding the strap back towards the caudal vertebra. The front section and rear section are then interconnected, e.g. by suturing, as illustrated in FIG. 16B by a black box. Consequently, it may be said that for the first loop, the first fastening edge and the second fastening edge are a caudal edge of the spinous process of the caudal vertebra and a cranial edge of the spinous process of the cranial vertebra, respectively. For the second loop, the first fastening edge and the second fastening edge are different, namely a hole in the spinous process of the caudal vertebra and a hole in the spinous process of the cranial vertebra, respectively.

In FIG. 16C, a further variant is shown which comprises one loop that includes two traverse positions at which the strap is passed from a first lateral side of the spinous process to an opposite second lateral side. More specifically, the traverse positions are arranged in holes in the cranial and caudal spinous process, respectively. The caudal edge of the spinous process of the caudal vertebra and the cranial edge of the spinous process of the cranial vertebra both act as fastening edges. The arrangement illustrated in FIG. 16C may be labelled as “double-8-arrangement”.

In FIG. 16D, no holes were drilled in the spinous processes of the two vertebrae and a double loop was formed around the caudal edge of the spinous process of the caudal vertebra and the cranial edge of the spinous process of the cranial vertebra. This arrangement may e.g. be labelled as “double-O-arrangement” or “double-O-no-hole-arrangement”.

In FIG. 16E, the caudal vertebra comprises a hole in its spinous process, while the cranial spinous process is devoid of holes. A double loop is formed, wherein for the cranial vertebra, the cranial edge of the spinous process was used as fastening edge for both loops, but for the caudal vertebra, the caudal edge of the spinous process was used for one of the two loops and the hole in the spinous process of the caudal vertebra was used for the other of the two loops. The arrangement illustrated in FIG. 16E may e.g. be labelled as “double-O-one-hole-arrangement”. It is understood that as an alternative, the hole may also be arranged in the cranial vertebra.

FIG. 17 shows a reinforcement structure that may be used to reinforce the fastening edge. In the illustrated embodiment, a metal sleeve is applied to a hole in the spinous process. The metal sleeve reinforces the hole, which may e.g. be used as fastening edge. Further, the metal sleeve includes smooth and rounded edges, which facilitate smooth gliding of the strap along its surface, in particular along its lateral edges, and minimize the risk of tearing of breaking of the strap. Furthermore, through its smooth surface, friction between the metal sleeve and the strap is minimized. The metal sleeve may also be easier for a surgeon to locate (e.g. using parallel imaging), thereby facilitating the medical procedure.

FIG. 18 shows further variants in which the strap passes through a loop formed by an intermediate section of the strap. More specifically, a loop formed by the intermediate section is arranged between the first vertebra and the second vertebra. The loop formed by the intermediate section of the strap may e.g. be used to enhance the stabilization of the two vertebrae with respect to each other. In particular, the loop may be used to control a degree of rotation of the two vertebrae with respect to an axis of rotation parallel to the caudal direction.

In FIG. 18A, the strap passes through the loop formed by the intermediate section of the strap two times. A further strap is eventually formed when the front section and the rear section of the strap are fastened to each other because the front section and the rear section are fastened to each other after extending across another section of the strap from opposite sides. In the variant shown in FIG. 18A, a single strap is used whose middle section forms the intermediate section.

In FIG. 18B, a variant is shown in which two straps are fastened to each other on their respective front sections as well as on their respective rear sections. The variant can also be viewed as a variant in which two separate straps are first fastened to each other to form a single unified strap. When viewed in this way, the variant shown in FIG. 18B is substantially similar to the variant shown in FIG. 18A, except that an additional fastening step is required to form the single unified strap in FIG. 18A.

In FIG. 18C, a variant is shown which comprises a single loop formed by the intermediate section of the strap. The strap passes through said loop two times. Eventually, the front section and the rear section of the strap are interconnected to each other on a position adjacent to the caudal vertebra.

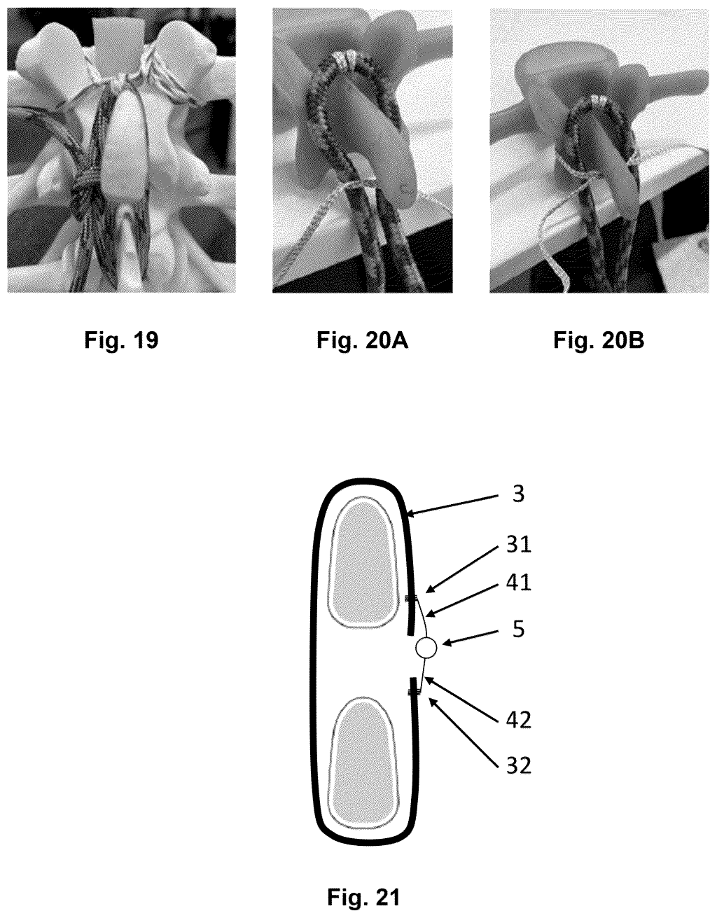

FIG. 19 shows a further variant in which a portion of the tension is shifted to another part of the spine. As illustrated in the figure, a tension distribution structure (illustrated by the cord) is used to interconnect (in a tensile-resistant manner) a cranial section of the strap to another part of the spine, namely in this example both superior articular processes of the cranial vertebra. More specifically, the section of the strap to which the tension distribution structure is interconnected is the section of the strap that contacts the cranial fastening edge. Thereby, at least a portion of the tensile force that would otherwise be applied fully to the cranial fastening edge is distributed to the superior articular processes instead. This reduces the wear and tear applied to the cranial spinous process. Furthermore, because the superior articular processes are arranged further in the ventral direction than the cranial fastening edge, the tension distribution structure also minimizes displacement of the strap in the dorsal direction.

FIG. 20 shows two variants featuring positional securing elements to secure the position of the strap with respect to displacement in the ventral or dorsal direction.

In FIG. 20A, a positional securing element (illustrated by a cord) is used to interconnect two intermediate sections of the strap that are arranged on opposite lateral sides of the spinous processes of the two vertebrae. By tying the two sections together, the strap is increasingly constricted around the cranial vertebra, which minimizes displacement of the strap, in particular with respect to displacement in the ventral or dorsal direction.

In FIG. 20B, a positional securing element (also illustrated by a cord) is used, which is passed around both intermediate sections of the strap that are arranged on opposite lateral sides of the spinous processes, and additionally around another structure of the spine, namely in this example the inferior articular process of the cranial vertebra. By tying the strap to the inferior articular process, displacement of the strap in the dorsal direction is minimized, or even essentially prevented.

FIG. 21 shows a further variant in which front and rear fastening fibers 41, 42 are used to interconnect the front end of the strap and the rear end of the strap 3. More specifically, a strap is looped around a cranial edge of the spinous process of the cranial vertebra and around a caudal edge of the spinous process of the caudal vertebra. Further, a front fastening fiber 41 is attached to the front end 31 of the strap, and a rear fastening fiber 42 is attached to a rear end 32 of the strap 3. In the illustrated example, the front and rear fastening fiber 41, 42 are each interconnected to the front and rear end 31, 32 of the strap 3 through a Prusik knot.

Upon tensioning, the front end 31 and the rear end 32 are approximated towards each other, wherein a gap is formed that interspaces the front end 31 and the rear end 32. The gap is bridged through interconnecting the front fastening fiber 41 and the rear fastening fiber 42. In the illustrated example, the front and rear fastening fibers 41, 42 are interconnected through a button 5, but other interconnection elements could also be used. It would also be possible to interconnect the front and rear fastening fibers 41, 42 in other ways, e.g. by directly knotting them together (not shown). In some variants, a single fastening fiber is used whose front end acts as front fastening fiber and whose rear end acts as rear fastening fiber.

### LIST OF REFERENCES

Park P, Garton H J, Gala V C, et al (2004) Adjacent Segment Disease after Lumbar or Lumbosacral Fusion&colon; Review of the Literature. Spine 29:1938-1944.
Chen Z, Zhao J, Xu H, et al (2008) Technical factors related to the incidence of adjacent superior segment facet joint violation after transpedicular instrumentation in the lumbar spine. Eur Spine J 17:1476-1480.
Marie-Hardy L, Pascal-Moussellard H, Barnaba A, et al (2020) Screw Loosening in Posterior Spine Fusion: Prevalence and Risk Factors. Global Spine J 10:598-602.
Ondra S L, Marzouk S (2003) Revision strategies for lumbar pseudarthrosis. Neurosurg Focus 15:1-5.
Chrastil J, Patel A A (2012) Complications Associated With Posterior and Transforaminal Lumbar Interbody Fusion. J Am Acad Orthop Sur 20:283-291.
Guan Y, Yoganandan N, Maiman D J, Pintar F A (2008) Internal and External Responses of Anterior Lumbar&sol; Lumbosacral Fusion&colon; Nonlinear Finite Element Analysis. J Spinal Disord Tech 21:299-304.
Kim H J, Iyer S (2016) Proximal Junctional Kyphosis. J Am Acad Orthop Sur 24:318-326.
Borgeaud T, Huec J-C L, Faundez A (2022) Pelvic and spinal postural changes between standing-sitting positions following lumbosacral fusion: a pilot study. Int Orthop 1-8.
Jung J-M, Hyun S-J, Kim K-J, Jahng T-A (2021) Dynamic Stabilization Surgery in Patients with Spinal Stenosis: Long-term Outcomes and the Future. Spine 46: E893-E900.
Prud'homme M, Barrios C, Rouch P, et al (2015) Clinical Outcomes and Complications After Pedicle-anchored Dynamic or Hybrid Lumbar Spine Stabilization. J Spinal Disord Techniques 28: E439-E448.
Cecchinato R, Bourghli A, Obeid I (2020) Revision surgery of spinal dynamic implants: a literature review and algorithm proposal. Eur Spine J 29:57-65.
Cornaz F, Fasser M-R, Spirig J M, et al (2019) 3D Printed Clamps Improve Spine Specimen Fixation in Biomechanical Testing. J Biomech 98:10946.
Krackow K A, Cohn B T (1987) A new technique for passing tendon through bone. Brief note. J Bone Joint Surg Am Volume 69:922-4.

1. A method of stabilizing the spine of a patient in need thereof, comprising:
a. providing a first vertebra (1) having a first fastening edge (11) and a second vertebra (2) having a second fastening edge (21), wherein the first fastening edge (11) and the second fastening edge (21) are arranged opposite each other and facing away from each other;
b. providing a strap (3) extending in a longitudinal direction from a front section (31) to a rear section (32);
c. passing the front section (31) of the strap (3) around the first fastening edge (11), then from the first fastening edge (11) to the second fastening edge (21) and then around the second fastening edge (21);
d. optionally passing the front section (31) of the strap (3) from the second fastening edge (21) to the first fastening edge (11) and repeating step c;
e. tensioning the strap (3) and fastening the front section (31) to the rear section (32) such that the first vertebra (1) and the second vertebra (2) are fixated relative to each other.

2. The method according to claim 1, wherein the strap is hyperelastic.

3. The method according to claim 1, wherein the step of fastening the front section (31) to the rear section (31) includes at least one of knotting, suturing, sewing, stitching, knitting or felting the front section (31) to the rear section (31).

4. The method according to claim 1, wherein the strap (3) comprises a non-metallic cerclage, a ligament or a tendon, preferably a tendon.

5. The method according to claim 1, wherein the strap (3) comprises a hybrid material comprising a ligament component and a synthetic components.

6. The method according to claim 1, wherein the strap (3) comprises a front fastening fiber (41) connected to a main body of the strap (3) in or near the front section (31) and a rear fastening fiber (42) connected to the main body of the strap (3) in or near the rear section (31).

7. The method of claim 6, wherein the rear fastening fiber (41) forms a loop configured for receiving the front fastening fiber (41).

8. The method according to claim 6, wherein the front fastening fiber (41) and/or the rear fastening fiber (41) are each connected to the main body of the strap (3) by a stitch, preferably a Krackow stitch, a Baseball stitch, a Prusik or a SpeedWhip.

9. The method according to claim 6, wherein the step of tensioning the strap (3) comprises creating a tension knot between the front fastening fiber (41) and the rear fastening fiber (41), tensioning at least the front fastening fiber (41) and tightening the tension knot.

10. The method according to claim 1, wherein one of the two vertebrae is a cranial vertebra and the other of the two vertebrae is a caudal vertebra, wherein the fastening edge of the cranial vertebra is a cranial edge of the lamina and the fastening edge of the caudal vertebra is a caudal edge of the lamina.

11. The method according to claim 10, wherein the distance in the longitudinal direction between a front end of the front section (31) and a front end of the rear section (31) of the strap (3) in a relaxed state ranges from 120% to 180%, preferably from 140% to 160%, more preferably 150%, of the distance between the first fastening edge (11) and the second fastening edge (11).

12. The method according to claim 1, wherein one of the two vertebrae is a cranial vertebra and the other of the two vertebrae is a caudal vertebra, wherein the fastening edge of the cranial vertebra is arranged on the spinous process of the cranial vertebra and/or the fastening edge of the caudal vertebra is arranged on the spinous process of the caudal vertebra.

13. The method according to claim 12, wherein the distance in the longitudinal direction between a front end of the front section (31) and a front end of the rear section (31) of the strap (3) in a relaxed state ranges from 300% to 500%, preferably from 350% to 450%, more preferably from 380% to 420%, of the distance between the first fastening edge (11) and the second fastening edge (11).

14. The method according to claim 1, wherein after fastening the front section (31) to the rear section (31), the strap (3) exerts a tensile force on the first vertebra (1) and on the second vertebra (2) in a tensile force direction that is essentially in parallel to the direction of extension of the spine between the first vertebra (1) and the second vertebra (2).

15. The method according to claim 1, wherein one of the two vertebrae is a cranial vertebra and the other of the two vertebrae is a caudal vertebra, wherein
a. either the fastening edge of the cranial vertebra is a cranial edge of the lamina of the cranial vertebra and the fastening edge of the caudal vertebra is arranged on the spinous process of the caudal vertebra;
b. or the fastening edge of the caudal vertebra is a caudal edge of the lamina of the caudal vertebra and the fastening edge of the cranial vertebra is arranged on the spinous process of the cranial vertebra.

16. The method according to claim 1, wherein after fastening the front section (31) to the rear section (31), the strap (3) exerts a tensile force on the first vertebra (1) and on the second vertebra (2) in a tensile force direction that includes a vector component in the dorsal direction.

17. The method according to claim 1, wherein during the step of tensioning the strap (3), a tension force from 40 N to 200 N, preferably from 50 N to 100 N, more preferably from 50 N to 70 N, is applied.

18. The method according to claim 1, wherein the step of fastening the front section (31) to the rear section (31) includes overlapping the front section (31) with the rear section (31) such that the front section (31) and the overlapped rear section (31) extend in the same or opposite directions, and connecting the front section (31) with the overlapped rear section (31).

19. The method according to claim 1, wherein after tensioning the strap (3), the front section and the rear section of the strap are interspaced from each other by a gap.

20. The method of claim 19, wherein the step of fastening the front section to the rear section comprises interconnecting a/the front fastening fiber to a/the rear fastening fiber, thereby bridging the gap.

21. The method according to claim 1, wherein step c) is repeated at least once, thereby forming at least a first loop defined in a first iteration of step c) and a second loop defined in a second iteration of step c).

22. The method according to claim 21, wherein the first loop and the second loop are interconnected at a loop interconnection position arranged in a spinal direction of extension between the spinous process of the cranial vertebra and the spinous process of the caudal vertebra.

23. The method according to claim 1, wherein at a position of the strap (3) in which the front section (31) has been fastened to the rear section (31), the strap (3) has a maximum width of up to 20 mm, preferably up to 15 mm, more preferably up to 10 mm, even preferably up to 8 mm, even more preferably up to 6 mm, in at least one of the following directions: dorsal direction or lateral di-rection.

24. The method according to claim 1, further comprising applying to the strap (3) a positional securing element configured for securing a position of the strap (3) with respect to displacement in a ventral or dorsal direction.

25. The method according to claim 1, further comprising inter-connecting the strap to a tension relief body structure through a tension distribution structure, such that at least a portion of the tension is distributed from the first and/or second fastening edge (11, 21) to the tension relief body structure.
